(19) United States
(12) Patent Application Publication
(10) Pub. No.: US 2017/0137880 A1
(43) Pub. Date: May 18, 2017

(54) METHOD FOR TREATING DEPRESSION AND MAJOR DEPRESSIVE DISORDER

(71) Applicant: Takeda Pharmaceutical Company Limited, Chuo-ku, Osaka-shi, Osaka (JP)

(72) Inventors: Jarlath Ffrench-Mullen, Deerfield, IL (US); Eric Lai, Deerfield, IL (US)

(73) Assignee: Takeda Pharmaceutical Company Limited, Chuo-ku, Osaka-shi, Osaka (JP)

(21) Appl. No.: 15/123,072

(22) PCT Filed: Mar. 4, 2015
(86) PCT No.: PCT/US15/18701

§ 371 Date: Sep. 1, 2016

Related U.S. Application Data
(60) Provisional application No. 61/948,529, filed on Mar. 5, 2014.
(60) Provisional application No. 62/061,417, filed on Oct. 8, 2014.

(51) Int. Cl. C12Q 1/68; A61K 31/495

(52) U.S. Cl. CPC C12Q 1/6883; A61K 31/495; C12Q 2600/156; C12Q 2600/106; C12Q 2600/16

(57) ABSTRACT

The present invention provides methods for treating depression such as major depressive disorder (MDD) in an individual. The invention further provides methods for determining if an individual suffering from depression is likely to respond favorably or experience an enhanced treatment effect in response to treatment with vortioxetine. The present invention also provides methods for treating cognitive impairment in an individual, optionally wherein the individual also suffers from depression and/or MDD. The invention further provides methods for determining if an individual suffering from cognitive impairment is likely to respond favorably or experience an enhanced treatment effect in response to treatment with vortioxetine. The methods comprise determining the presence of polymorphisms in the collagen, type XXVI, alpha 1 (COL26A1) gene and/or the calcium channel, voltage-dependent, L type, alpha 1C subunit (CACNA1C) gene and/or the CUB and Sushi Multiple Domains 1 (CSMD1) gene and/or the Zinc Finger Protein 494 (ZSCAN4) gene and/or the Zinc Finger Protein 551 (ZNF551) gene and/or the dymeclin (DYM) gene and/or the LINC00348 gene and/or the FOXL2NB gene and/or intergenic regions in the individual.

### CROSS-REFERENCE TO RELATED APPLICATIONS

The present application claims priority to and the benefit of U.S. Provisional Patent Application Nos. 61/948,529 filed Mar. 5, 2014 and 62/061,417 filed Oct. 8, 2014. The foregoing provisional application is incorporated by reference herein in its entirety.

### FIELD

The present invention relates to methods and kits for treating depression such as major depressive disorder (MDD) in an individual, and for identifying the likelihood that an individual suffering from depression such as MDD will respond favorably to treatment with vortioxetine and/or experience an enhanced treatment effect when treated with vortioxetine. These methods and kits are based on detecting the presence of polymorphisms in the collagen, type XXVI, alpha 1 (COL26A1) gene and/or the calcium channel, voltage-dependent, L type, alpha 1C subunit (CACNA1C) gene and/or the CUB and Sushi Multiple Domains 1 (CSMD1) gene and/or the Zinc Finger Protein 494 (ZSCAN4) gene and/or the Zinc Finger Protein 551 (ZNF551) gene and/or the dymeclin (DYM) gene and/or the LINC00348 gene and/or the FOXL2NB gene and/or intergenic regions.

### BACKGROUND

Depression is a state of low mood and aversion to activity that can affect a person's thoughts, behavior, feelings and sense of well-being. A depressed person may feel sad, anxious, empty, hopeless, worried, helpless, worthless, guilty, irritable, hurt, or restless. A number of psychiatric syndromes feature depressed mood as a main symptom. Mood disorders are a group of disorders considered to be primary disturbances of mood, such as major depressive disorder (MDD; commonly called major depression or clinical depression) where a person has at least two weeks of depressed mood or a loss of interest or pleasure in nearly all activities.

More specifically, major depressive disorder (MDD) is a disabling, severe mental disorder characterized by episodes of all-encompassing low mood accompanied by low self-esteem and loss of interest or pleasure in normally enjoyable activities. The illness tends to be chronic and repeated episodes are common. Other symptoms of MDD may include irritability or frustration, sleep disturbances, tiredness and lack of energy, changes in appetite, anxiety, agitation, restlessness, feelings of worthlessness or guilt, trouble thinking and concentrating, and unexplained physical problems, such as back pain or headaches. The disorder is a significant contributor to the global burden of disease and affects people in all communities across the world (Ferrari, 2013). MDD is a highly prevalent psychiatric disorder with twin studies revealing that up to 40% of MDD cases are genetically determined (Kendler, 2006). Although the exact causes of MDD are unknown, it is believed that a variety of factors may be involved, such as brain chemistry and physical brain differences, hormones, inherited traits and life events.

Many types of antidepressant medications are available to treat MDD and other mood disorders that present with depression. Some available drugs include selective serotonin reuptake inhibitors (SSRIs), serotonin and norepinephrine reuptake inhibitors (SNRIs), norepinephrine and dopamine reuptake inhibitors (NDRIs), tricyclic antidepressants, monoamine oxidase inhibitors (MAOIs), and atypical antidepressants such as vortioxetine. However, despite the availability of numerous treatment options, individual response to antidepressant medication is suboptimal and variable. That is, not all individuals respond equally to a given antidepressant. As many as one half of patients do not receive adequate treatment of MDD and many respond partially or not at all to treatment.

Vortioxetine is a bis-aryl-sulfanyl amine psychotropic indicated for the treatment of MDD. Although its mechanism of antidepressant effect is not fully understood, vortioxetine is known to enhance serotonergic activity in the central nervous system by inhibiting serotonin reuptake (e.g., by acting as a 5-HT receptor antagonist). This activity is believed to influence the antidepressive effect of vortioxetine. Vortioxetine also has several other activities including 5-HT3 receptor antagonism and 5-HT1A receptor agonism. However, the contribution of these activities to vortioxetine's antidepressant effect has not been established.

It is believed that inherited traits may play a role in how an antidepressant affects an individual but other variables besides genetics can also affect response to medication. As a result, it is not easy to predict which medication is the best treatment option for a given patient. Accordingly, it would be beneficial to devise a method for identifying subpopulations of patients suffering from depression and/or MDD that are likely to respond most favorably to a particular MDD medication such as vortioxetine.

### SUMMARY

One aspect of the present invention provides a method for treating depression and/or MDD in an individual identified as (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive, the method comprising administering vortioxetine to the individual.

Another aspect of the invention provides a method for treating depression and/or MDD in an individual, comprising determining the individual is (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive, and administering vortioxetine to the individual.

Another aspect of the present invention provides a method for determining the likelihood that an individual suffering from depression and/or MDD will respond favorably to treatment with vortioxetine. Some aspects comprise obtaining a biological sample from the individual. Some aspects comprise assaying a biological sample from the individual for the presence of COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual. Some aspects comprise determining that the individual is likely to respond favorably to treatment with vortioexetine when the individual is (i) homozygous for COL26A1 rs4045; (ii) possesses a CACNA1C variant; (iii) possesses a CSMD1 variant, (iv) possesses a ZSCAN4 variant, (v) possesses a ZNF551 variant, (vi) is homozygous for COL26A1 rs4045 and possesses a CACNA1C variant, (vii) is homozygous for COL26A1 rs4045 and possesses a CACNA1C variant and a CSMD1 variant, or (viii) is homozygous for COL26A1 rs4045 and possesses a CACNA1C variant, a CSMD1 variant, and a ZSCAN4 variant, (ix) is homozygous for COL26A1 rs4045 and possesses a CACNA1C variant, a CSMD1 variant, a ZSCAN4 variant, and a ZNF551 variant, (x) is homozygous for COL26A1 rs4045 and possesses a CACNA1C variant, a CSMD1 variant, a ZSCAN4 variant, a DYM variant, and an intergenic variant, or (x) is homozygous for COL26A1 rs4045 and possesses a CACNA1C variant, a CSMD1 variant, a ZSCAN4 variant, a DYM variant, a LINC00348 variant, a FOXL2NB variant, and an intergenic variant.

In some embodiments, the methods further comprise assaying the biological sample to determine the presence of COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual, and determining that the individual is likely to respond favorably to treatment with vortioxetine when the individual is (i) homozygous for COL26A1 rs4045; (ii) possesses a CACNA1C variant; or (iii) possesses a CSMD1 variant, (iv) possesses a ZSCAN4 variant, (v) possesses a ZNF551 variant (vi) is homozygous for COL26A1 rs4045 and possesses a CACNA1C variant (vii) is homozygous for COL26A1 rs4045 and possesses a CACNA1C variant and a CSMD1 variant, (viii) is homozygous for COL26A1 rs4045 and possesses a CACNA1C variant, a CSMD1 variant, and a ZSCAN4 variant, (ix) is homozygous for COL26A1 rs4045 and possesses a CACNA1C variant, a CSMD1 variant, a ZSCAN4 variant, and a ZNF551 variant, (x) is homozygous for COL26A1 rs4045 and possesses a CACNA1C variant, a CSMD1 variant, a ZSCAN4 variant, a DYM variant, and an intergenic variant, or (ix) is homozygous for COL26A1 rs4045 and possesses a CACNA1C variant, a CSMD1 variant, a ZSCAN4 variant, a DYM variant, a LINC00348 variant, a FOXL2NB variant, and an intergenic variant. In some embodiments the individual having a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant is determined to be homozygous for the variant.

Yet another aspect of the present invention provides a method for determining the likelihood that an individual suffering from depression and/or MDD will experience an enhanced treatment effect when treated with vortioxetine comprising assaying a biological sample from the individual for the presence or absence of COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual; and determining if the individual is likely to experience an enhanced treatment effect when treated with vortioxetine when the COL26A1 rs4045 and/or the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant are detected in the sample.

In some embodiments of the methods of the invention, the individual suffers from and/or has been clinically diagnosed with major depressive disorder (MDD).

In some embodiments of the present invention, vortioxetine can be used to treat an individual with cognitive impairment wherein the individual is determined or has been identified to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. Some embodiments comprise a method for determining the likelihood that an individual suffering from cognitive impairment will experience an enhanced treatment effect when treated with vortioxetine when the individual is determined to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. Likewise, also described herein is a method for determining the likelihood that an individual suffering from cognitive impairment will respond favorably to treatment with vortioxetine when the individual is (i) homozygous for COL26A1 rs4045 positive, (ii) CACNA1C variant positive, (iii) CSMD1 variant positive, (iv) ZSCAN4 variant positive, (v) ZNF551 variant positive, (vi) homozygous for COL26A1 rs4045 and CACNA1C variant positive, (vii) homozygous for COL26A1 rs4045 and CACNA1C and CSMD1 variant positive, (viii) homozygous for COL26A1 rs4045 and CACNA1C, CSMD1, and ZSCAN4 variant positive, (ix) homozygous for COL26A1 rs4045 and CACNA1C, CSMD1, ZSCAN4, and ZNF551 variant positive, (x) homozygous for COL26A1 rs4045 and CACNA1C, CSMD1, ZSCAN4, DYM, and intergenic variant positive, or (xi) homozygous for COL26A1 rs4045 and CACNA1C, CSMD1, ZSCAN4, DYM, LINC00348, FOXL2NB and intergenic variant positive. In some aspects of this invention, the disclosed methods contemplate treating with vortioxetine to improve cognitive function. In some embodiments, the individual being treated, responding favorably to treatment, and/or experiencing an enhanced treatment effect is identified as homozygous for COL26A1 rs4045 and CACNA1C variant positive, CSMD1 variant positive, and ZSCAN4 variant positive.

In some aspects, the individual with cognitive impairment also suffers from or has been diagnosed with depression and/or MDD. In some aspects, the disclosed methods contemplate treating with vortioxetine an individual diagnosed with depression and/or MDD to improve cognitive function.

In some embodiments, the disclosed methods comprise determining that the individual is heterozygous for the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant. In other embodiments the methods comprise determining that the individual is homozygous for the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant. In some embodiments, the disclosed methods comprise determining that the individual is homozygous for COL26A1 rs4045.

In some embodiments, the CACNA1C variant is selected from the group consisting of rs7297992, rs7297582 (position 2355806, alleles C/T), rs2239042 (position 2428487, alleles G/A), rs3819532, rs2239079, rs2239080, kgp5074525, rs4765961, kgp1052923, kgp1390211, rs7311147 (position 2707821, alleles G/A), rs12312322, rs2108636, rs2238043, rs7295089, kgp3964892, rs10848664, kgp2586442, rs4765700, rs2238095, rs12312322, rs7972947, rs10848664, rs1006737 (position 2345295, alleles G/A), rs2370602, and combinations thereof. In a particular embodiment, the CACNA1C variant is selected from the group consisting of rs7297582 (position 2355806, alleles C/T), rs2239042, rs7311147 (position 2707821, alleles G/A), and combinations thereof.

In some embodiments, the CSMD1 variant is rs59420002.

In some embodiments, the ZSCAN4 variant is selected from the group consisting of rs9304796, rs73064580, rs12983596, rs12984275, rs9749513, rs12609579, rs4239480, rs9676604, rs12162232, and combinations thereof.

In some embodiments, the ZNF551 variant is rs12162230.

In some embodiments, the DYM variant is rs62104612.

In some embodiments, the LINC00348 variant is rs145136593.

In some embodiments, the FOXL2NB variant is rs116191388.

In some embodiments, the intergenic variant is selected from the group consisting of rs1998609, rs4142192, and combinations thereof.

The methods of the invention may comprise assaying a sample from the individual to determine the presence of the rs4045 variant and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in the genome of the individual. The sample may be selected from the group consisting of a body fluid, a tissue sample, cells, and isolated nucleic acids. A sample of isolated nucleic acids may comprise DNA and/or RNA from the individual. In some embodiments, assaying a sample from the individual involves reverse transcribing the RNA to produce cDNA.

One aspect of the present invention provides kits, such as kits comprising (i) at least one pair of primers that specifically hybridizes to a genetic variant as disclosed herein and (ii) a detectably labeled probe that hybridizes to the genetic variant. In some embodiments, the genetic variant is independently selected from the group consisting of rs4045, rs59420002, rs7297582, rs2239042, and rs7311147.

In some embodiments, the kits comprise a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; and a pair of primers that specifically hybridizes to rs9749513.

In some embodiments, the kits comprise a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; a pair of primers that specifically hybridizes to rs9749513; a pair of primers that specifically hybridizes to rs62104612; a pair of primers that specifically hybridizes to rs1998609; and a pair of primers that specifically hybridizes to rs4142192.

In some embodiments, the kits comprise a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; a pair of primers that specifically hybridizes to rs9749513; a pair of primers that specifically hybridizes to rs62104612; a pair of primers that specifically hybridizes to rs1998609; a pair of primers that specifically hybridizes to rs145136593; and a pair of primers that specifically hybridizes to rs116191388.

In some embodiments, the kits comprise a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs9304796; a pair of primers that specifically hybridizes to 73064580; a pair of primers that specifically hybridizes to rs12983596; a pair of primers that specifically hybridizes to rs12984275; a pair of primers that specifically hybridizes to rs9749513; a pair of primers that specifically hybridizes to rs12609579; a pair of primers that specifically hybridizes to rs4239480; a pair of primers that specifically hybridizes to rs9676604; and a pair of primers that specifically hybridizes to rs12162232.

### BRIEF DESCRIPTION OF THE DRAWINGS

FIG. 1 shows a summary of the genotype of individuals in the study after Quality Control (QC) procedures. QC procedures evaluated variant cell rate (per variant, using all samples), minor allele frequency (per variant, using all samples) and Hardy-Weinberg Equilibrium test p-values (per variant, using all samples per race).

FIG. 2 provides a summary of statistics after Quality Control.

FIG. 3 summarizes the subgroup identification results. MDD individuals with rs4045 and a CACNA1C variant experience a significantly enhanced response to treatment with vortioxetine as measured by MADRS and HAM-A scores.

FIG. 4 provides graphic results of the data obtained from the vortioxetine vs. placebo subgroup identification study.

FIG. 5 depicts least square (LS) means plots for the MADRS scores (5(B)), HAM-A scores (5(C)) and overall response (RESP) scores (5(D)) for individuals with the specified rs1006737 alleles (GG=homozygous for the G allele; GA=heterozygous; and AA=homozygous for the A). For each plot graph (B through D), the 3 left-most data points represent the treatment groups (20 mg vortioxetine) and the 3 right-most data points represent placebo groups.

FIG. 6 depicts least square (LS) means plots for the MADRS scores (6(B)), HAM-A scores (6(C)) and overall response (RESP) scores (6(D)) for individuals with the specified rs7297582 alleles (CC=homozygous for the C allele; CT=heterozygous; and TT=homozygous for the T allele). For each plot graph (B through D), the 3 left-most data points represent the treatment groups (20 mg vortioxetine) and the 3 right-most data points represent placebo groups.

FIG. 7 depicts least square (LS) means plots for the MADRS scores (7(B)), HAM-A scores (7(C)) and overall response (RESP) scores (7(D)) for individuals with the specified rs2239042 alleles (AA=homozygous for the A allele; AG=heterozygous; and GG=homozygous for the G allele). For each plot graph (B through D), the 3 left-most data points represent the treatment groups (20 mg vortioxetine) and the 3 right-most data points represent placebo groups.

FIG. 8 depicts least square (LS) means plots for the MADRS scores (8(B)), HAM-A scores (8(C)) and overall response (RESP) scores (8(D)) for individuals with the specified rs7311147 alleles (AA=homozygous for the A allele; AG=heterozygous; and GG=homozygous for the G allele). For each plot graph (B through D), the 3 left-most data points represent the treatment groups (20 mg vortioxetine) and the 3 right-most data points represent placebo groups.

FIG. 9 provides graphic or tabulated results of the data obtained from (i) a vortioxitene vs. placebo 5-variant model subgroup identification study for all subjects (9(A)), white non-hispanic subjects (9(B)), and all subjects in an initial 426 subject subgroup (9(C)); (ii) a summary of baseline and demographic characteristics for subjects in the studies using the 5-variant model (9(D)); (iii) responder and remission rates at week 8 determined by LOCF (9E and 9F); (iv) changes from baseline in MADRS scores in the 5-variant model after treatment with 20 mg vortioxetine vs. placebo (9G-I), (v) CGI-I scores in the 5-variant model (9J), (vi) change from baseline in HAM-A total scores at week 8 for both the 5-variant model (FIG. 9K), (vii) responder rates based on race in the 5-variant model (FIG. 9L), (viii) nausea rates following 20 mg vortioxitene administration in the 5-variant model (9M), (ix) a vortioxitene vs. duloxitene vs. placebo 5-variant model subgroup identification study for all subjects (9(N)); and (iii) an overall response status plot for individuals with A/G EMID2 alleles (9(O)), A/G rs2239042 alleles (9(P)), C/T rs7297582 alleles (9(Q)), A/G rs1006737 alleles (9(R)), and A/G rs7311147 alleles (9(S)). In the figures PK<LLQ indicates that the pharmacokinetics were below the lower limit of quantification.

FIG. 10 provides graphic or tabulated results of the data obtained from (i) a vortioxitene vs. placebo 7-variant model subgroup identification study for all subjects (10(A)), white non-hispanic subjects (10(B)), and all subjects in an initial 426 subject subgroup (10(C)) and (ii) a vortioxitene vs. placebo 5-variant model subgroup identification study for all subjects (10(D)), (iii) a summary of baseline and demographic characteristics for subjects in the studies using the 7-variant model (10D), (iv) responder and remission rates at week 8 determined by LOCF (10E and 10F), (v) changes from baseline in MADRS scores in the 7-variant model after treatment with 20 mg vortioxetine vs. placebo (10G-I), (vi) a comparison of the change in from baseline in MADRS total score in the 5-variant model and 7-variant model is shown in (10J), (vii) CGI-I scores in the 7-variant model (10K), (viii) change from baseline in HAM-A total scores at week 8 for both the 7-variant model (10L), (ix) responder rates based on race in the 7-variant model (10M), and (x) a vortioxitene vs. duloxitene vs. placebo 7-variant model subgroup identification study for all subjects (10N)

FIG. 11 provides graphic results of the data obtained from the vortioxitene vs. placebo 14-variant model subgroup identification study for all subjects.

FIG. 12 provides graphic results of the data obtained from a 10 mg vortioxetine vs. 20 mg vortioxetine vs. placebo 5-variant and 7-variant model subgroup identification study (i.e., TAK-316 study), and the Figure includes a graphic or tabulated representation of (i) a 10 mg vortioxetine vs. 20 mg vortioxetine vs. placebo 7-variant model subgroup identification study (12A and 12D), (ii) a 10 mg vortioxetine vs. placebo 7-variant model subgroup identification study (12B), (iii) a 10 mg vortioxetine vs. 20 mg vortioxetine vs. placebo 5-variant model subgroup identification study (12C), and (iv) sample accountability data (12E).

FIG. 13 shows improvement in cognition, as measured by a Cognitive and Physical Functioning Questionnaire (CPFQ), following administration of 10 mg/day and 20 mg/day vortioxetine relative to administration of a placebo. The results are tabulated in FIG. 13A and graphically represented in FIG. 13B. FIG. 13C shows a graphical representation of the 10 mg/day and 20 mg/day vortioxetine data calculated relative to placebo data.

FIG. 14 shows a change from baseline in MADRS total score in a 10-variant model subgroup identification study following administration of (i) 60 mg duloxetine and 20 mg vortioxetine relative to a placebo (14A and 14B), (ii) 10 mg vortioxetine and 20 mg vortioxetine relative to a placebo (14C and 14D), and (iii) 10 mg vortioxetine relative to a placebo (14E and 14F).

FIG. 15 provides graphic results of data obtained from a 11-variant model subgroup identification study and shows (i) changes from baseline in MADRS scores following administration of 20 mg vortioxetine, 10 mg vortioxetine, and/or 60 mg duloxetine (15A-D) and (ii) a change from baseline in MADRS total score following administration of (a) 60 mg duloxetine and 20 mg vortioxetine relative to a placebo (15E and 15F), (ii) 10 mg vortioxetine and 20 mg vortioxetine relative to a placebo (15G and 15H), and (iii) 10 mg vortioxetine relative to a placebo (15I and 15J).

### DETAILED DESCRIPTION

Provided herein are methods for treating depression such as major depressive disorder (MDD) in an individual suffering from depression or MDD. In some embodiments, the individual has been clinically diagnosed with depression or a depression-related mood disorder such as MDD. Also described herein are methods for identifying individuals suffering from depression such as MDD who will likely respond favorably to treatment with vortioxetine. Methods for identifying individuals suffering from depression such as MDD who will likely experience an enhanced treatment response to vortioxetine as compared to another individual are also described.

Also provided herein are methods for treating cognitive impairment in an individual suffering from cognitive impairment. Also described herein are methods for identifying individuals suffering from cognitive impairment who will likely respond favorably to treatment with vortioxetine. Methods for identifying individuals suffering from cognitive impairment who will likely experience an enhanced treatment response to vortioxetine as compared to another individual are also described. In some embodiments, the individual suffering from cognitive impairment also suffers from depression and/or MDD.

Target Population

The present inventors surprisingly discovered that individuals suffering from MDD who are homozygous for COL26A1 rs4045 and possess a CACNA1C variant, a CSMD1 variant, a ZSCAN4 variant, a ZNF551 variant, a DYM variant, a LINC00348 variant, a FOXL2NB variant, and/or an intergenic variant are more likely to experience an enhanced treatment response to vortioextine than individuals who are not homozygous for COL26A1 rs4045 and who do not possess a CACNA1C variant, a CSMD1 variant, a ZSCAN4 variant, a ZNF551 variant, a DYM variant, a LINC00348 variant, a FOXL2NB variant, and/or an intergenic variant. Individuals with this genotype are likely to respond favorably to treatment with vortioxetine, meaning, the individual experiences a ≧50% improvement in their MADRS score in response to a particular treatment regimen administered to mitigate the depression as compared to their baseline score. In some embodiments, the treatment is administration of vortioextine.

As used herein, “COL26A1” refers to the collagen, type XXVI, alpha 1 gene, which is located on chromosome 7 in humans. COL26A1 is also known as EMID2. The transcription start and end positions are located at 101,006,001 and 101,202,304, respectively. An exemplary gene sequence for COL26A1 is Gene ID: 136227, the sequence of which is incorporated by reference herein.

As used herein, a “COL26A1 rs4045” variant or polymorphism describes the single nucleotide polymorphism present in the COL26A1 gene at chromosome 7, position 101067089. A portion of a COL26A1 gene sequence that comprises the rs4045 is as follows:

(SEQ ID NO: 1)
TGTTCTGTTTTGGCCTCCGAACTCC[A/G]AGGAGTGAGTGAGAAGAAC
TCCCTG

wherein the [A/G] signifies the variation. An individual who possesses at least one copy of the COL26A1 rs4045 variant is referred to as “COL26A1 rs4045 positive” or “rs4045 positive.” Additional COL26A1 variants include kgp3405169 (chromosome 7 position 101,071,257, allele=A, allele=G), and rs6949799 (chromosome 7 position 101,067,526, allele=C, allele=T). In some embodiments, the COL26A1 variant is selected from the group consisting of rs4045, rs6949799, kpg3405169, and combinations thereof.

As used herein, the “CACNA1C” gene refers to the calcium channel, voltage-dependent, L type, alpha 1C subunit gene with the cytogenetic location at 12p13.3 (i.e., located on the short (p) arm of chromosome 12 at position 13). The transcription start and stop positions, based on Genome Reference Consortium human genome assembly version 38 (GRCh38), are located at 1,970,786 and 2,697,949, respectively. The calcium channel produced from the CACNA1C gene is known as CaV1.2. These channels are found in many types of cells, although they appear to be particularly important for the normal function of heart and brain cells. An exemplary nucleotide sequence of the CACNA1C gene is that of NCBI Gene ID: 775, the sequence of which is incorporated by reference herein. Over 23 transcript variants have been reported that result from this gene and exemplary cDNA sequences corresponding to various mRNA transcripts are known and publicly available.

Over 528 SNPs have been identified in the CACNA1C gene. As used herein, a “CACNA1C variant” is a CACNA1C gene with a sequence that is less than 100% identical to that of NCBI Gene ID: 775. In some embodiments, the variant has a sequence identity that is from about 75% to about 99% identical to that of NCBI Gene ID: 775, such as about 75%, about 80%, about 85%, about 90%, about 95%, or about 99% identical to that of NCBI Gene ID: 775. Accordingly, a CACNA1C gene with a single nucleotide variation from the sequence of NCBI Gene ID: 775 is a CACNA1C variant. In some embodiments, a CACNA1C variant is a CACNA1C polynucleotide that exhibits at least one polymorphism in the CACNA1C coding region as compared to the coding region of NCBI Gene ID: 775. In some embodiments, a CACNA1C variant that is associated with an individual's response to vortioxetine is a CACNA1C missense mutation (also known as a nonsynonymous mutation).

In some embodiments, a CACNA1C variant that is associated with a favorable response to vortioxetine or an enhanced treatment effect is located within partition 3 (position 2333638-2436522) or partition 6 (position 2538549-2565920). In some embodiments, a CACNA1C variant that is associated with a favorable response to vortioxetine or an enhanced treatment effect is selected from the group consisting of rs7297992, rs7297582 (position 2355806, alleles C/T), rs2239042 (position 2428487, alleles G/A), rs3819532, rs2239079, rs2239080, kgp5074525, rs4765961, kgp1052923, kgp1390211, rs7311147 (position 2707821, alleles G/A), rs12312322, rs2108636, rs2238043, rs7295089, kgp3964892, rs10848664, kgp2586442, rs4765700, rs2238095, rs12312322, rs7972947, rs10848664, rs1006737 (position 2345295, alleles G/A), rs2370602, and combinations thereof. In a particular embodiment, the CACNA1C variant is selected from the group consisting of rs7297582 (position 2355806, alleles C/T), rs2239042, rs7311147 (position 2707821, alleles G/A), and combinations thereof. In some embodiments, a CACNA1C variant that is associated with a favorable response to vortioxetine or an enhanced treatment effect is selected from the group consisting of rs7297582, rs2239042, rs7311147, and combinations thereof.

An individual who is heterozygous or homozygous for a CACNA1C variant is “CACNA1C variant positive.”

As used herein, the “CSMD1” gene refers to CUB and Sushi Multiple Domains 1 protein gene, which is located on chromosome 8. The transcription start and end positions, based on GRCh38, are located at 2,935,353 and 4,994,806, complement. An exemplary gene sequence for CSMD1 is NCBI Gene ID: 64478, the sequence of which is incorporated by reference herein.

As used herein, “CSMD1 variant” is a CSMD1 gene with a sequence that is less than 100% identical to that of NCBI Gene ID: 64478. In some embodiments, the variant has a sequence identity that is from about 75% to about 99% identical to that of NCBI Gene ID: 64478, such as about 75%, about 80%, about 85%, about 90%, about 95%, or about 99% identical to that of NCBI Gene ID: 64478. In some embodiments, a CSMD1 variant is a CSMD1 polynucleotide that exhibits at least one polymorphism in the CSMD1 coding region as compared to the coding region of NCBI Gene ID: 64478. In some embodiments, a CSMD1 variant is associated with a favorable response to vortioxetine. In some embodiments, a CSMD1 variant that is associated with a favorable response to vortioxetine is rs59420002 (alleles A/G).

An individual who is heterozygous or homozygous for a CSMD1 variant is “CSMD1 variant positive.”

As used herein, the “ZSCAN4” gene refers to Zinc Finger Protein 494 gene, which is located on chromosome 19. The transcription start and end positions, based on GRCh38, are located at 57,668,935 and 57,679,152. An exemplary gene sequence for ZSCAN4 is NCBI Gene ID: 201516, the sequence of which is incorporated by reference herein.

As used here, “ZSCAN4 variant” is a ZSCAN4 gene with a sequence that is less than 100% identical to that of NCBI Gene ID: 201516. In some embodiments, the variant has a sequence identity that is from about 75% to about 99% identical to that of NCBI Gene ID: 201516, such as about 75%, about 80%, about 85%, about 90%, about 95%, or about 99% identical to that of NCBI Gene ID: 201516. In some embodiments, a ZSCAN4 variant is a ZSCAN4 polynucleotide that exhibits at least one polymorphism in the ZSCAN4 coding region as compared to the coding region of NCBI Gene ID: 201516. In some embodiments, a ZSCAN4 variant is associated with a favorable response to vortioxetine. In some embodiments, a ZSCAN4 variant that is associated with a favorable response to vortioxetine is selected from the group consisting of rs9304796 (alleles G/T), rs73064580 (alleles C/T), rs12983596 (alleles C/T), rs12984275 (alleles C/G), rs9749513 (alleles C/T), rs12609579 (alleles A/C), rs4239480 (alleles A/G), rs9676604 (alleles C/T), rs12162232 (alleles A/G), rs10417057 (alleles T/C), rs10403851 (alleles G/A), rs56066537 (alleles G/T), rs112783430 (alleles G/T), rs9749360 (alleles A/G), and combinations thereof.

An individual who is heterozygous or homozygous for a ZSCAN4 variant is “ZSCAN4 variant positive.”

As used herein, the “ZNF551” gene refers to Zinc Finger Protein 551 gene, which is located on chromosome 19. The transcription start and end positions, based on GRCh38, are located at 57,681,969 and 57,689,811. An exemplary gene sequence for ZNF551 is NCBI Gene ID: 90233, the sequence of which is incorporated by reference herein.

As used here, “ZNF551 variant” is a ZNF551 gene with a sequence that is less than 100% identical to that of NCBI Gene ID: 90233. In some embodiments, the variant has a sequence identity that is from about 75% to about 99% identical to that of NCBI Gene ID: 90233, such as about 75%, about 80%, about 85%, about 90%, about 95%, or about 99% identical to that of NCBI Gene ID: 90233. In some embodiments, a ZNF551 variant is a ZNF551 polynucleotide that exhibits at least one polymorphism in the ZNF551 coding region as compared to the coding region of NCBI Gene ID: 90233. In some embodiments, a ZNF551 variant is associated with a favorable response to vortioxetine. In some embodiments, a ZNF551 variant that is associated with a favorable response to vortioxetine is rs12162230 (alleles G/A).

An individual who is heterozygous or homozygous for a ZNF551 variant is “ZNF551 variant positive.”

As used herein, the “DYM” gene refers to the dymeclin gene, which is located on chromosome 18. The transcription start and end positions, based on GRCh38, are located at 49,043,026 and 49,460,709. An exemplary gene sequence is NCBI Gene ID: 54808, the sequence of which is incorporated by reference herein.

As used herein, “DYM variant” is a DYM gene with a sequence identity that is less than 100% identical to that of NCBI Gene ID: 54808. In some embodiments, the variant has a sequence identity that is from about 75% to about 99% identical to that of NCBI Gene ID: 54808, such as about 75%, about 80%, about 85%, about 90%, about 95%, or about 99% identical to that of NCBI Gene ID: 54808. In some embodiments, a DYM variant is a DYM polynucleotide that exhibits at least one polymorphism in the DYM coding region as compared to the coding region of NCBI Gene ID: 54808. In some embodiments, a DYM variant is associated with a favorable response to vortioxetine. In some embodiments, a DYM variant that is associated with a favorable response to vortioxetine is rs62104612.

An individual who is heterozygous or homozygous for a DYM variant is “DYM variant positive.”

As used herein, the “LINC00348” gene refers to the Long Intergenic Non-Protein Coding RNA 348 gene, which is located on chromosome 13. The transcription start and end positions, based on GRCh38, are located at 71,143,183 and 71,168,417. An exemplary gene sequence is NCBI Gene ID: 100885781, the sequence of which is incorporated by reference herein.

As used herein, “LINC00348 variant” is a LINC00348 gene with a sequence identity that is less than 100% identical to that of NCBI Gene ID: 100885781. In some embodiments, the variant has a sequence identity that is from about 75% to about 99% identical to that of NCBI Gene ID: 100885781, such as about 75%, about 80%, about 85%, about 90%, about 95%, or about 99% identical to that of NCBI Gene ID: 100885781. In some embodiments, a LINC00348 variant is a LINC00348 polynucleotide that exhibits at least one polymorphism in the LINC00348 coding region as compared to the coding region of NCBI Gene ID: 100885781. In some embodiments, a LINC00348 variant is associated with a favorable response to vortioxetine. In some embodiments, a LINC00348 variant that is associated with a favorable response to vortioxetine is rs145136593.

An individual who is heterozygous or homozygous for a LINC00348 variant is “LINC00348 variant positive.”

As used herein, the “FOXL2NB” gene (also known as the C3orf72 gene) refers to the FOXL2 neighbor gene, which is located on chromosome 3. The transcription start and end positions, based on GRCh38, are located at 138,947,234 and 138,953,988. An exemplary gene sequence is NCBI Gene ID: 401089, the sequence of which is incorporated by reference herein.

As used herein, “FOXL2NB variant” is a FOXL2NB gene with a sequence identity that is less than 100% identical to that of NCBI Gene ID: 401089. In some embodiments, the variant has a sequence identity that is from about 75% to about 99% identical to that of NCBI Gene ID: 401089, such as about 75%, about 80%, about 85%, about 90%, about 95%, or about 99% identical to that of NCBI Gene ID: 401089. In some embodiments, a FOXL2NB variant is a FOXL2NB polynucleotide that exhibits at least one polymorphism in the FOXL2NB coding region as compared to the coding region of NCBI Gene ID: 401089. In some embodiments, a FOXL2NB variant is associated with a favorable response to vortioxetine. In some embodiments, a FOXL2NB variant that is associated with a favorable response to vortioxetine is rs116191388.

An individual who is heterozygous or homozygous for a FOXL2NB variant is “FOXL2NB variant positive.”

As used herein, “intergenic region” refers to a region between two genes. As used herein, “intergenic variant” is a region between two genes that exhibits at least one polymorphism as compared to a wild type intergenic region. In some embodiments, an intergenic variant is associated with a favorable response to vortioxetine. In some embodiments, an intergenic variant that is associated with a favorable response to vortioxetine is selected from the group consisting of rs1998609, rs4142192, and combinations thereof.

An individual who is heterozygous or homozygous for an intergenic variant is “intergenic variant positive.”

As used herein, an individual who suffers from MDD is an individual who meets the Diagnostic and Statistical Manual of Mental Disorders (DSM-IV-TR) criteria for MDD. In some embodiments, an individual who suffers from MDD has experienced a major depressive episode (MDE) for at least 3 months. In some embodiments, the individual has a MADRS total score of ≧26, and/or a CGI-S score of ≧4 prior to treatment.

Depression symptoms and the degree of improvement experienced with treatment are assessed using standard depression symptom rating scales such as the Hamilton Depression Rating Scale (HAM-A), the Montgomery-Asberg Depression Rating Scale (MADRS), and/or the Clinical Global Impression Improvement psychological scale (CGI scale). Treatment efficacy is determined based on an improvement in one or more depressive symptoms as measured by mean change in HAM-A total score, MADRS total score, and/or CGIS total score from baseline.

As used herein, an individual who experiences an “enhanced treatment response” to a drug such as vortioextine experiences a greater improvement in depression symptoms when treated than an individual suffering from depression and/or MDD who has been treated but is not homozygous for COL26A1 rs4045 and who is not homozygous for the rs7297582, rs2239042, or rs7311147 CACNA1C variant; and/or the rs59420002 CSMD1 variant; and/or the rs9304796, rs73064580, rs12983596, rs12984275, rs9749513, rs12609579, rs4239480, rs9676604, rs12162232, rs10417057, rs10403851, rs56066537, rs112783430, or rs9749360 ZSCAN4 variant; and/or the rs12162230 ZNF551 variant; and/or the rs62104612 DYM variant; and/or the rs145136593 LINC00348 variant; and/or the rs116191388 FOXL2NB variant; and/or the rs1998609 or rs4142192 intergenic variant.

Methods of Treatment

In one embodiment, a method for treating depression and/or MDD in an individual identified as (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB and intergenic variant positive as provided herein comprises administering vortioxetine to the individual.

In another embodiment, a method for treating depression and/or MDD in an individual comprises determining the individual is (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive and administering vortioxetine or a similar bis-aryl-sulfanyl amine psychotropic to the individual.

Vortioxetine may be administered or ingested in a tablet form that contains vortioxetine HBr (1-[2-(2,4-Dimethyl-phenylsulfanyl)-phenyl]-piperazine, hydrobromide) having the structural formula

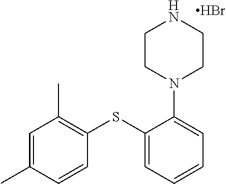 [SMILES: Br ; Cc1ccc(Sc2ccccc2N2CCNCC2)c(C)c1]

In some embodiments, vortioxetine is administered or ingested at a dose of 5, 10, 15, or 20 mg per day. In some embodiments the starting dose is 10 mg/day, which is ultimately increased to 20 mg/day. Treatment may continue for at least 5, 6, 7, or 8 weeks. Oral administration is preferred, but other administration modes may be employed. Given that the patient population identified in the present invention is likely to experience enhanced treatment effect in response to vortioxetine, lower dosages may be administered. For example, dosages less than 5 mg per day may be administered, such as dosages of 2.5, 3, 3.5, 4 or 4.5 mg per day.

In a further embodiment, a method for determining the likelihood that an individual suffering from depression and/or MDD is likely to experience an enhanced treatment effect when treated with vortioxetine is disclosed. In some embodiments, the method comprises assaying a sample from the individual suffering from depression and/or MDD to determine the presence or absence of a COL26A1 rs4045 variant and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual. The individual is determined to be likely to experience an enhanced treatment effect when treated with vortioxetine if the COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant are present in nucleic acids from the individual.

### Methods of Predicting Response to Vortioxetine

A method for determining the likelihood that an individual suffering from depression and/or MDD will respond favorably to treatment with vortioxetine is also provided herein. In some embodiments, the method comprises assaying a sample from the individual to determine the presence of the COL26A1 rs4045 variant and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual, and determining that the individual is likely to respond favorably to treatment with vortioxetine when the individual is homozygous for CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant.

In some embodiments, the methods of the invention further comprise administering vortioxetine to the COL26A1 rs4045, CACNA1C variant positive, CSMD1 variant positive, ZSCAN4 variant positive, ZNF551 variant positive, DYM variant positive, LINC00348 variant positive, FOXL2NB variant positive, and/or intergenic variant positive individual.

In some embodiments, determining an individual is COL26A1 rs4045 positive and/or CACNA1C variant positive and/or CSMD1 variant positive and/or ZSCAN4 variant positive and/or ZNF551 variant positive and/or DYM variant positive and/or LINC00348 variant positive and/or FOXL2NB variant positive and/or intergenic variant positive involves obtaining a biological sample from an individual and assaying the sample to determine the presence of a COL26A1 rs4045 and/or a CACNA1C sequence variant and/or a CSMD1 sequence variant and/or a ZSCAN4 sequence variant and/or a ZNF551 sequence variant and/or a DYM sequence variant and/or a LINC00348 sequence variant and/or a FOXL2NB sequence variant and/or an intergenic sequence variant in the genome of the individual.

The sample that is assayed may be a sample of any substance obtained from an individual wherein the substance contains nucleic acids from the individual. Exemplary sample types include a body fluid sample, a tissue sample, a stool sample, cells from the individual, and isolated nucleic acids obtained from the individual. Exemplary body fluid samples include blood, plasma, serum, cerebrospinal fluid, and saliva. Exemplary tissue samples include tissue biopsy samples. Exemplary cell samples include buccal swabs or cells obtained from any biological samples taken from the individual. Methods of extracting nucleic acids from samples are well known in the art and can be readily adapted to obtain a sample that is compatible with the system utilized. Automated sample preparation systems for extracting nucleic acids from a test sample are commercially available, e.g., Roche Molecular Systems' COBAS AmpliPrep System, Qiagen's BioRobot 9600, and Applied Biosystems' PRISM™ 6700 sample preparation system.

As used herein, “isolated nucleic acids” denotes nucleic acids that are removed to at least some extent from the cellular material from which they originated. However “isolated” does not require that the nucleic acids are completely pure and free of any other components. Examples of isolated nucleic acids are those obtained using commercial nucleic extraction kits.

In some embodiments, a sample from an individual contains DNA and/or RNA from the individual. In some embodiments, assaying a sample involves extracting nucleic acids from a biological sample to determine that the individual is COL26A1 rs4045 positive and CACNA1C variant positive and/or CSMD1 variant positive and/or ZSCAN4 variant positive and/or ZNF551 variant positive and/or DYM variant positive and/or LINC00348 variant positive and/or FOXL2NB variant positive and/or intergenic variant positive. Various methods of extraction are suitable for isolating DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield DNA and/or RNA. However, nucleic extraction is not essential and a sample such as, for example, blood or saliva may be assayed directly to determine that the individual is COL26A1 rs4045 positive and CACNA1C variant positive and/or CSMD1 variant positive and/or ZSCAN4 variant positive and/or ZNF551 variant positive and/or DYM variant positive and/or LINC00348 variant positive and/or FOXL2NB variant positive and/or intergenic variant positive without extracting nucleic acids from the sample.

In some embodiments, assaying a sample comprises reverse transcribing RNA to produce cDNA.

In some embodiments, assaying a sample comprises amplifying nucleic acids in the sample or nucleic acids derived from nucleic acids in the sample (e.g. cDNA). Amplification methods which may be used include variations of RT-PCR, including quantitative RT-PCR, for example as adapted to the method described by Wang, A. M. et al., Proc. Natl. Acad. Sci. USA 86:9717-9721, (1989), or by Karet, F. E., et al., Analytical Biochemistry 220:384-390, (1994). Another method of nucleic acid amplification or mutation detection which may be used is ligase chain reaction (LCR), as described by Wiedmann, et al., PCR Methods Appl. 3:551-564, (1994). An alternative method of amplification or mutation detection is allele specific PCR (ASPCR). ASPCR which utilizes matching or mismatching between the template and the 3′ end base of a primer well known in the art. See e.g., U.S. Pat. No. 5,639,611, which is incorporated herein by reference and made a part hereof.

Another method of assaying a sample to determine the presence of a genetic variant comprises nucleic acid sequencing. Sequencing can be performed using any number of methods, kits or systems known in the art. One example is using dye terminator chemistry and an ABI sequencer (Applied Biosystems, Foster City, Calif.). Sequencing also may involve single base determination methods such as single nucleotide primer extension (“SNapShot®” sequencing method) or allele or mutation specific PCR. The SNaPshot® Multiplex System is a primer extension-based method that enables multiplexing up to 10 SNPs (single nucleotide polymorphisms). The chemistry is based on the dideoxy single-base extension of an unlabeled oligonucleotide primer (or primers). Each primer binds to a complementary template in the presence of fluorescently labeled ddNTPs and AmpliTaq® DNA Polymerase, FS. The polymerase extends the primer by one nucleotide, adding a single ddNTP to its 3′ end. SNaPshot® Multiplex System is commercially available (ABI PRISM. SNaPshot® Multiplex kit, Applied Biosystems Foster City, Calif.). Products generated using the ABI PRISM® SNaPshot® Multiplex kit can be analyzed with GeneScan® Analysis Software version 3.1 or higher using ABI PRISM® 310 Genetic Analyzer, ABI PRISM® 3100 Genetic Analyzer or ABI PRISM® 3700 DNA Analyzer.

A person skilled in the art will recognize that, based on the SNP and associated sequence information disclosed herein, detection reagents can be developed and used to assay any SNP of the present technology individually or in combination, and that such detection reagents can be incorporated into a kit.

The term “kit” as used herein in the context of SNP detection reagents, refers to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.).

Accordingly, the present technology further provides SNP detection kits and systems, including but not limited to, packaged probe and primer sets (e.g., TaqMan probe/primer sets), arrays/microarrays of nucleic acid molecules, and beads that contain one or more probes, primers, or other detection reagents for detecting one or more SNPs described herein. The kits can optionally include various electronic hardware components. For example, arrays (“DNA chips”) and microfluidic systems (“lab-on-a-chip” systems) provided by various manufacturers typically comprise hardware components. Some kits (e.g., TaqMan probe/primer sets) may not include electronic hardware components, but may be comprised of, for example, one or more SNP detection reagents (along with other optional biochemical reagents) packaged in one or more containers.

In some embodiments, a SNP detection kit contains one or more detection reagents and other components (e.g., buffers, reagents, enzymes having polymerase activity, enzymes having polymerase activity and lacking 5′→3′ exonuclease activity or both 5′→3′ and 3′→5′ exonuclease activity, ligases, enzyme cofactors such as magnesium or manganese, salts, chain extension nucleotides such as deoxynucleoside triphosphates (dNTPs) or biotinylated dNTPs, and in the case of Sanger-type DNA sequencing reactions, chain terminating nucleotides (i.e., dideoxynucleoside triphosphates (ddNTPs), positive control sequences, negative control sequences, and the like) to carry out an assay or reaction, such as amplification and/or detection of a SNP-containing nucleic acid molecule. In some embodiments, a kit contains a means for determining the amount of a target nucleic acid, determining whether an individual is heterozygous or homozygous for a polymorphism or when detecting a gene transcript, and/or comparing the amount with a standard. In some embodiments, the kit comprises instructions for using the kit to detect the SNP-containing nucleic acid molecule of interest. In some embodiments, the kits contain reagents to carry out one or more assays to detect one or more SNPs disclosed herein. In some embodiments, SNP detection kits are in the form of nucleic acid arrays or compartmentalized kits, including microfluidic/lab-on-a-chip systems.

The kits may further comprise one or more of: wash buffers and/or reagents, hybridization buffers and/or reagents, labeling buffers and/or reagents, and detection means. The buffers and/or reagents can be optimized for the particular amplification/detection technique for which the kit is intended. Protocols for using these buffers and reagents for performing different steps of the procedure may also be included in the kit.

In some embodiments, the SNP detection kit comprises at least one set of primers (e.g., comprising one matched allele-specific primer and one mismatched allele-specific primer) and, optionally, a non-extendable oligonucleotide probe. Each kit can comprise reagents which render the procedure specific. Thus, a kit intended to be used for the detection of a particular SNP can comprise a matched and mismatched allele-specific primers set specific for the detection of that particular SNP, and optionally, a non-extendable oligonucleotide probe. A kit intended to be used for the multiplex detection of a plurality of SNPs comprises a plurality of primer sets, each set specific for the detection of one particular SNP, and, optionally, a plurality of corresponding non-extendable oligonucleotide probes.

In some embodiments, the SNP detection kit comprises multiple pairs of primers for one or more target SNP loci, wherein said primers are designed so that the lengths of said PCR products from different SNP loci or from different alleles of the same SNP locus are sufficiently distinguishable from each other in capillary electrophoresis analysis, thus making them suitable to multiplex PCR. The SNP detection kit can further comprise a fluorescently labeled single-base extension/termination reagent, i.e., ddNTPs, to label the primers during the multiplex PCR reaction (e.g., SNaPshot Multiplex). The chemistry of the SNP detection kit can be based on the dideoxy single-base extension of the unlabeled primers. Each primer can bind to its target SNP loci in the presence of fluorescently labeled ddNTPs and the polymerase extends the primer by one nucleotide, adding a single ddNTP to its 3′ end. The identity of the incorporated nucleotide can be determined by the fluorescence color readout. In some embodiments, the kits comprise multiple pairs of primers for simultaneously detecting at least one SNP locus having two or more different alleles. In some embodiments, the kits comprise multiple pairs of primers for simultaneously detecting different genotypes among 1-8 different SNP loci. In some embodiments, the SNP detection kit comprises multiple pairs of primers that have the annealing temperatures designed to be used in a single amplification reaction. In some embodiments, the kits further comprise an internal control polynucleotide and/or multiple control primers for conducting multiplex PCR using the internal control polynucleotide as a template.

In some embodiments, SNP detection kits may contain, for example, one or more probes, or pairs of probes, that hybridize to a nucleic acid molecule at or near each target SNP position. Multiple pairs of allele-specific probes may be included in the kit to simultaneously assay multiple SNPs, at least one of which is a SNP disclosed herein. In certain embodiments, multiple pairs of allele-specific probes are included in the kit to simultaneously assay all of the SNPs described herein. In some embodiments, the kit includes capture primers and optionally extension primers for the detection of one or a plurality of SNPs of one or more genes selected from the group consisting of COL26A1, CACNA1C, CSMD1, ZSCAN4, ZNF551, DYM, LINC00348 and FOXL2NB.

In some embodiments, the SNP detection kits comprise at least one set of pre-selected nucleic acid sequences that act as capture probes for the extension products. The pre-selected nucleic acid sequences (allele-specific probes) may be immobilized on an array or beads (e.g., coded beads), and can be used to detect at least 1, 4, 10, 11, all, or any combination of the SNPs disclosed herein. By way of example only, the kits may include polystyrene microspheres that are internally dyed with two spectrally distinct fluorescent dyes (e.g., x-MAP™ microbeads, Luminex Corp. (Austin, Tex.)). Using precise ratios of these fluorophores, a large number of different fluorescent bead sets can be produced (e.g., a set of 100). Each set of beads can be distinguished by its code (or spectral signature) and can be used to detect a large number of different extension products in a single reaction vessel. These sets of fluorescent beads with distinguishable codes can be used to label extension products. Labeling (or attachment) of extension products to beads can be by any suitable means including, but not limited to, chemical or affinity capture, cross-linking, electrostatic attachment, and the like. In some embodiments, labeling of extension products is carried out through hybridization of the allele-specific primers and the tag probe sequences. The magnitude of the biomolecular interaction that occurs at the microsphere surface is measured using a third fluorochrome that acts as a reporter (e.g., biotinylated dNTPs). Because each of the different extension products is uniquely labeled with a fluorescent bead, the captured extension product (indicative of one allele of a SNP of interest) can be distinguishable from other different extension products (including extension products indicative of other alleles of the same SNP and extension products indicative of other SNPs of interest). Following hybridization, the microbeads can be analyzed using methods such as flow cytometry. In embodiments where the primer extension reaction is carried out in the presence of biotinylated dNTPs, the reaction between beads and extension products may be quantified by fluorescence after reaction with fluorescently-labeled streptavidin (e.g., Cy5-streptavidin conjugate) using instruments such as the Luminex® 100™ Total System, Luminex® 100™ IS Total System, Luminex™ High Throughput Screening System).

Some embodiments provide methods of identifying the SNPs disclosed herein in a biological sample comprising incubating a test sample of nucleic acids obtained from the subject with an array comprising one or more probes corresponding to at least one SNP position disclosed herein, and assaying for binding of a nucleic acid from the test sample with one or more of the probes. Conditions for incubating a test sample with a SNP detection reagent from a kit that employs one or more such SNP detection reagents can vary. Incubation conditions depend on factors such as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

In some embodiments, the SNP detection kits of the present technology include control analytes for spiking into a sample, buffers, including binding, washing and elution buffers, solid supports, such as beads, protein A or G or avidin coated sepharose or agarose, etc., and a matrix-assisted laser desorption/ionization (MALDI) sample plate. The kit may also contain a database, which may be a table, on paper or in electronic media, containing information for one or a plurality of SNPs of one or more genes selected from the group consisting of COL26A1, CACNA1C, CSMD1, ZSCAN4, ZNF551, DYM, LINC00348 and FOXL2NB. In some embodiments, the kits contain programming to allow a robotic system to perform the present methods, e.g., programming for instructing a robotic pipettor or a contact or inkjet printer to add, mix and remove reagents. The various components of the kit may be present in separate containers or certain compatible components may be precombined into a single container, as desired.

In some embodiments, the kits include one or more other reagents for preparing or processing an analyte sample for matrix-assisted laser desorption/ionization time-of-flight mass spectrometry (MALDI-TOF). The reagents may include one or more matrices, solvents, sample preparation reagents, buffers, desalting reagents, enzymatic reagents, denaturing reagents, where calibration standards such as positive and negative controls may be provided as well. As such, the kits may include one or more containers such as vials or bottles, with each container containing a separate component for carrying out a sample processing or preparing step and/or for carrying out one or more steps of a MALDI-TOF protocol.

In addition to above-mentioned components, the kits include instructions for using the components of the kit to prepare a MALDI-TOF sample plate and/or assess a sample. The instructions, such as for preparing or assessing a sample via MALDI-TOF, are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate. In addition to the database, programming and instructions, the kits may also include one or more control analyte mixtures, e.g., two or more control samples for use in testing the kit.

Next generation sequencing (NGS) also may be used to determine an individual's genotype. Next generation sequencing is a high throughput, massively parallel sequencing method (e.g., one that uses a large number of processors or separate computers) that can generate multiple sequencing reactions of clonally amplified molecules and of single nucleic acid molecules in parallel. This allows increased throughput and yield of data. NGS methods include, for example, sequencing-by-synthesis using reversible dye terminators, and sequencing-by-ligation. Non-limiting examples of commonly used NGS platforms include miRNA BeadArray (Illumina, Inc.), Roche 454™ GS FLX™-Titanium (Roche Diagnostics), XMAP (Luminex Corp.), IONTORRENT™ (Life Technologies Corp.) and ABI SOLiD™ System (Applied Biosystems, Foster City, Calif.).

### Cognition

In one embodiment of the present invention, vortioxetine can be used to treat an individual with cognitive impairment identified to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. In another embodiment, a method for determining the likelihood that an individual suffering from cognitive impairment will experience an enhanced treatment effect when treated with vortioxetine when the individual is determined to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. Likewise, also described herein is a method for determining the likelihood that an individual suffering from cognitive impairment will respond favorably to treatment with vortioxetine when the individual is determined to be (i) homozygous for COL26A1 rs4045 positive, (ii) CACNA1C variant positive, (iii) CSMD1 variant positive, (iv) ZSCAN4 variant positive, (v) ZNF551 variant positive, (vi) homozygous for COL26A1 rs4045 and CACNA1C variant positive, (vii) homozygous for COL26A1 rs4045 and CACNA1C and CSMD1 variant positive, (viii) homozygous for COL26A1 rs4045 and CACNA1C, CSMD1, and ZSCAN4 variant positive, (ix) homozygous for COL26A1 rs4045 and CACNA1C, CSMD1, ZSCAN4, and ZNF551 variant positive, (x) homozygous for COL26A1 rs4045 and CACNA1C, CSMD1, ZSCAN4, DYM, and intergenic variant positive, or (xi) homozygous for COL26A1 rs4045 and CACNA1C, CSMD1, ZSCAN4, DYM, LINC00348, FOXL2NB and intergenic variant positive. In some aspects of this invention, the disclosed methods contemplate treating with vortioxetine an individual diagnosed with impaired cognitive function. In a most preferred embodiment, the individual being treated, responding favorably to treatment, and/or experiencing an enhanced treatment effect, the individual is identified as homozygous for COL26A1 rs4045 and CACNA1C variant positive, CSMD1 variant positive, and ZSCAN4 variant positive.

In one embodiment of the present invention, vortioxetine can be used to treat an individual with cognitive impairment, wherein the individual suffers from depression and/or MDD and is determined or has been identified to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. In another embodiment, a method for determining the likelihood that an individual suffering from cognitive impairment, wherein the individual also suffers from depression and/or MDD, will experience an enhanced treatment effect when treated with vortioxetine when the individual is determined to be (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive. Likewise, also described herein is a method for determining the likelihood that an individual suffering from cognitive impairment will respond favorably to treatment with vortioxetine, when the individual suffers from despression and/or MDD and is determined to be (i) homozygous for COL26A1 rs4045 positive, (ii) CACNA1C variant positive, (iii) CSMD1 variant positive, (iv) ZSCAN4 variant positive, (v) ZNF551 variant positive, (vi) homozygous for COL26A1 rs4045 and CACNA1C variant positive, (vii) homozygous for COL26A1 rs4045 and CACNA1C and CSMD1 variant positive, (viii) homozygous for COL26A1 rs4045 and CACNA1C, CSMD1, and ZSCAN4 variant positive, (ix) homozygous for COL26A1 rs4045 and CACNA1C, CSMD1, ZSCAN4, and ZNF551 variant positive, (x) homozygous for COL26A1 rs4045 and CACNA1C, CSMD1, ZSCAN4, DYM, and intergenic variant positive, or (xi) homozygous for COL26A1 rs4045 and CACNA1C, CSMD1, ZSCAN4, DYM, LINC00348, FOXL2NB and intergenic variant positive. In some aspects of this invention, the disclosed methods contemplate treating with vortioxetine an individual diagnosed with depression and/or MDD to improve cognitive function. In a most preferred embodiment, the individual being treated, responding favorably to treatment, and/or experiencing an enhanced treatment effect, the individual is identified as homozygous for COL26A1 rs4045 and CACNA1C variant positive, CSMD1 variant positive, and ZSCAN4 variant positive.

According to the CDC, cognitive impairment is when a person has trouble remembering, learning new things, concentrating, or making decisions that affect their everyday life. Cognitive impairment ranges from mild to severe. With mild impairment, people may begin to notice changes in cognitive functions, but still be able to do their everyday activities. Severe levels of impairment can lead to losing the ability to understand the meaning or importance of something and the ability to talk or write, resulting in the inability to live independently. Cognitive ability can be assessed by, for example, Massachusetts General Hospital Cognitive and Physical Functioning Questionnaire (see, e.g., Fava et al., J. Clin. Psychiatry, 67:11 (2006)) and/or the Digit Symbol Substitution Test (DSST) (see Mahableshwarker et al., Neuropsychopharmacology, in press 2015).

Cognitive disorders are a common type of neurological disorders. For example, dementia is form of impaired cognition caused by brain dysfunction. The hallmark of Alzheimer's Dementia (as well as some other forms of dementia) is the disruption of memory performance. Among the several conditions labeled as dementia, the most common are Alzheimer's disease and mild cognitive impairment (MCI), which is a pre-clinical form of Alzheimer's disease, as well as Parkinson's Disease Dementia and Lewy Body dementia. As described herein, cognitive impairment is associated with schizophrenia, attention deficit hyperactive disorder, bipolar disorder post stroke cognitive deficits, closed head injury, post-operative cognitive deficits, Huntington's Disease, generalized anxiety disorder (GAD), and post-traumatic stress disorder (PTSD). Some embodiments comprise treating cognitive impairment associated with diseases or conditions disclosed herein.

### REFERENCES

Fava M, Graves L M, Benazzi F, Scalia M J, Iosifescu D V, Alpert J E,& Papakostas G I. A cross-sectional study of the prevalence of cognitive and physical symptoms during long-term antidepressant treatment, J. Clin. Psychiatry, 67:11 (2006)
Ferrari, A J, Charlson F J, Norman R E, Flaxman A D, Patten S B, Vos T & Whiteford H A The Epidemiological Modelling of Major Depressive Disorder: Application for the Global Burden of Disease Study 2010. PlosOne 8(7):e69637, 2013.
Kendler K S, Gatz M, Gardner C O and Pedersen N L. A Swedish National Twin Study of Lifetime Major Depression. Am J Psychiatry 163:109-114, 2006.
Mahableshwarker, A R, Zajecka, J, Jacobsen W, Chen Y & Keefe R S A Randomized, Placebo-Controlled, Active-Reference, Double-Blind, Flexible-Dose Study of the Efficacy of Vortioxetine on Cognitive Function in Major Depressive Disorder. Neuropharmacology, Fen. 17, 2015; in press.

The subject matter of all references disclosed in this application are incorporated herein in their entirety.

### EXAMPLES

### Example 1

### Study Design—Treatment Groups

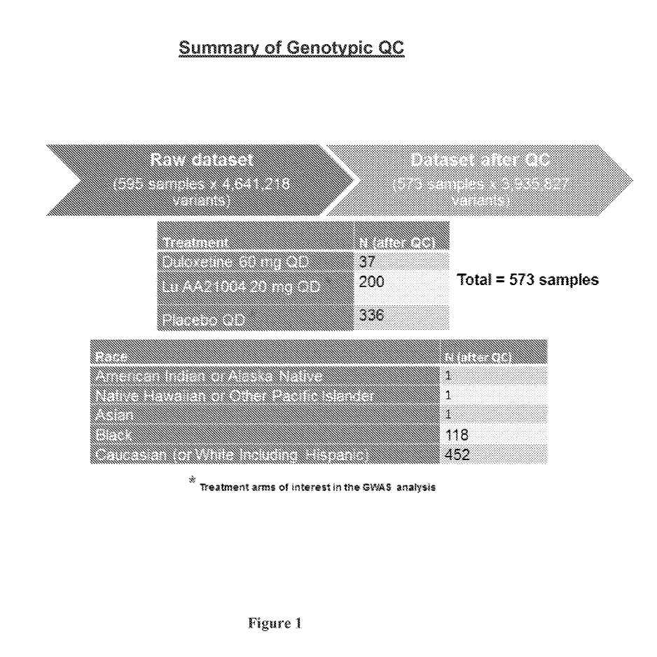

Multicenter, randomized, double-blind, parallel-group, placebo-controlled, drug-referenced, fixed-dose studies were conducted to evaluate the efficacy and safety of vortioxetine (10, 15 and 20 mg/Day) in the acute treatment of adult patients with MDD. A total of 595 individuals meeting the diagnostic criteria from the DSM-IV-TR for recurrent MDD were included in the studies. The current major depressive disorder for each individual was confirmed by the Structured Clinical Interview for DSM Disorders (SCID). The individuals had a reported duration of their current MDE of at least 3 months. The individuals also had a total MADRS score of ≧26 and a CGI-S score of ≧4 at the screening and baseline visits. Individuals were treated with 10, 15 or 20 mg vortioxetine, or a different drug, or a placebo daily for 8 weeks. See FIG. 1.

Subjects were seen weekly during the first 2 weeks of treatment and then every 2 weeks up to the end of the 8-week treatment period. The primary outcome measure was change from baseline in MADRS total score after 8 Weeks of treatment. The Montgomery Asberg Depression Rating Scale (MADRS) is a depression rating scale consisting of 10 items, each rated 0 (no symptom) to 6 (severe symptom). The 10 items represent the core symptoms of depressive illness. The rating is based on a clinical interview with the patient, moving from broadly phrased questions about symptoms to more detailed ones, which allow a precise rating of severity, covering the most recent 7 days. Total score is from 0 to 60, with a higher the score being the more severe.

Secondary outcome measures included the proportion of responders at week 8 (responders defined as a 50% decrease in MADRS total score from baseline); a change from baseline in MADRS total score at week 8; and a change in clinical status using CGI-I score at week 8. The Clinical Global Impression—Global Improvement (CGI-I) scale is a 7-point scale rated from 1 (very much improved) to 7 (very much worse). The investigator rated the patient's overall improvement relative to baseline, whether or not, in the opinion of the investigator, this was entirely due to the drug treatment.

### Study Design—Genotype Determination

Nucleic acid samples from the individuals were run on an Illumina™ HumanOmni5EXOME whole genome bead-chip array according to the manufacturer's protocol. The raw dataset of 595 samples times 4,641,218 variants was narrowed to 473 samples times 3,923,897 variants following quality control (QC). See FIG. 1.

### Analysis

A composite score (or genetic score) was calculated via penalized regression using a set of focused genomic regions. The cutoff point of the genetic score that maximizes treatment specific effect was then identified. Statistical significance (via a parametric bootstrap approach) of the treatment effect for the subgroup defined by the optimal cutpoint was then determined.

### Results

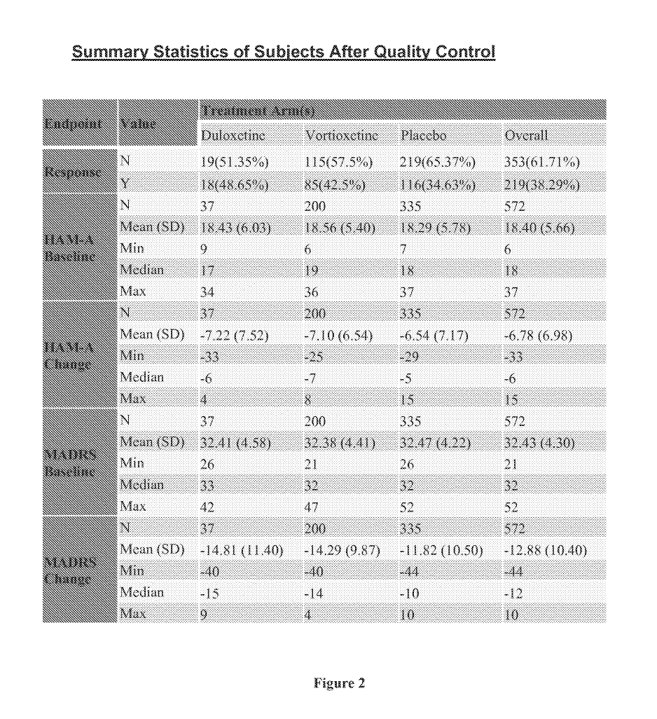

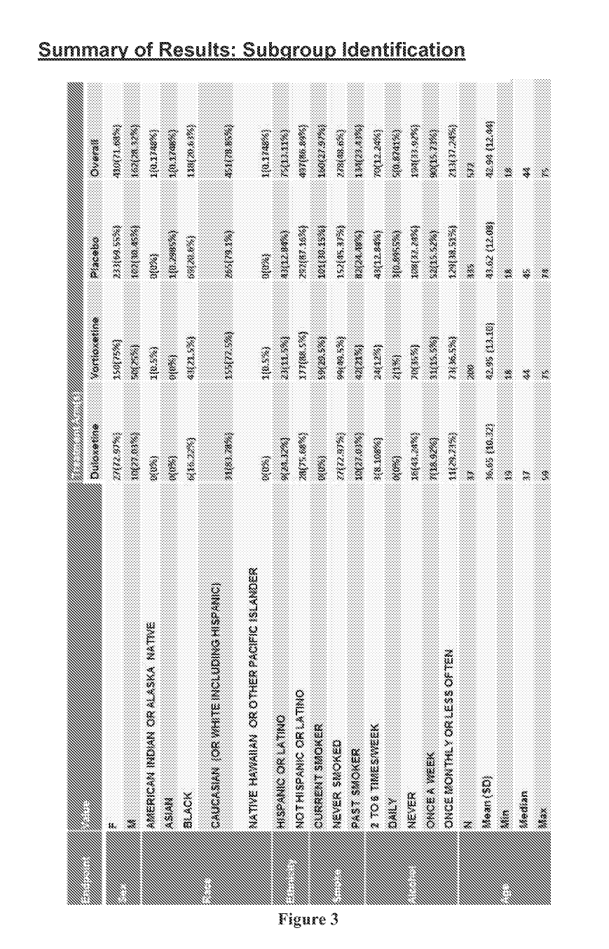

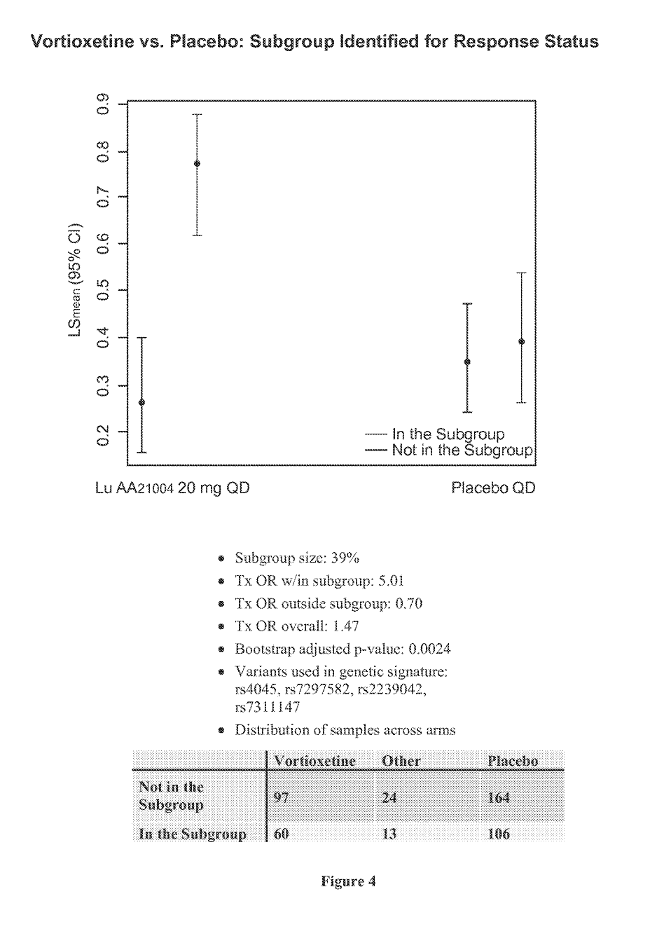

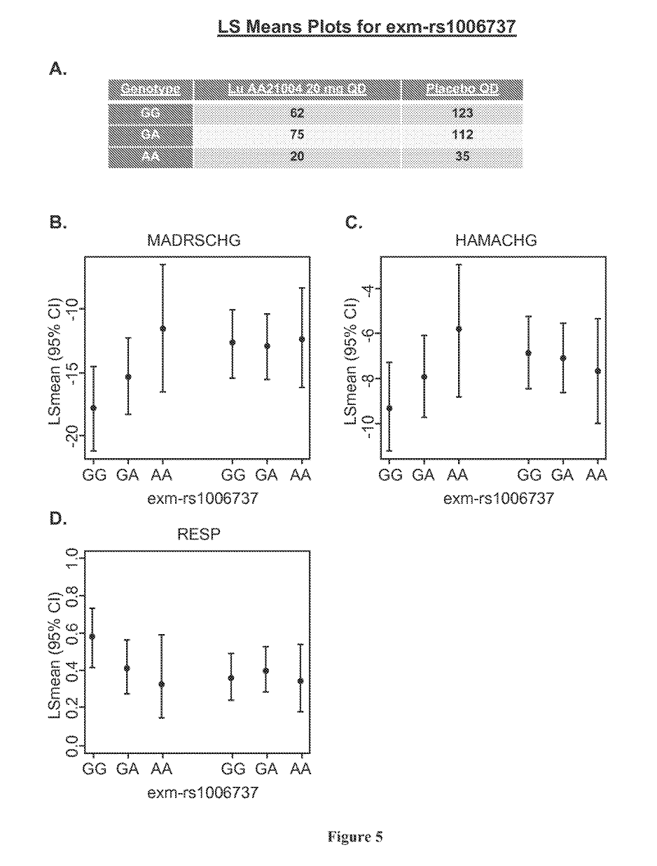

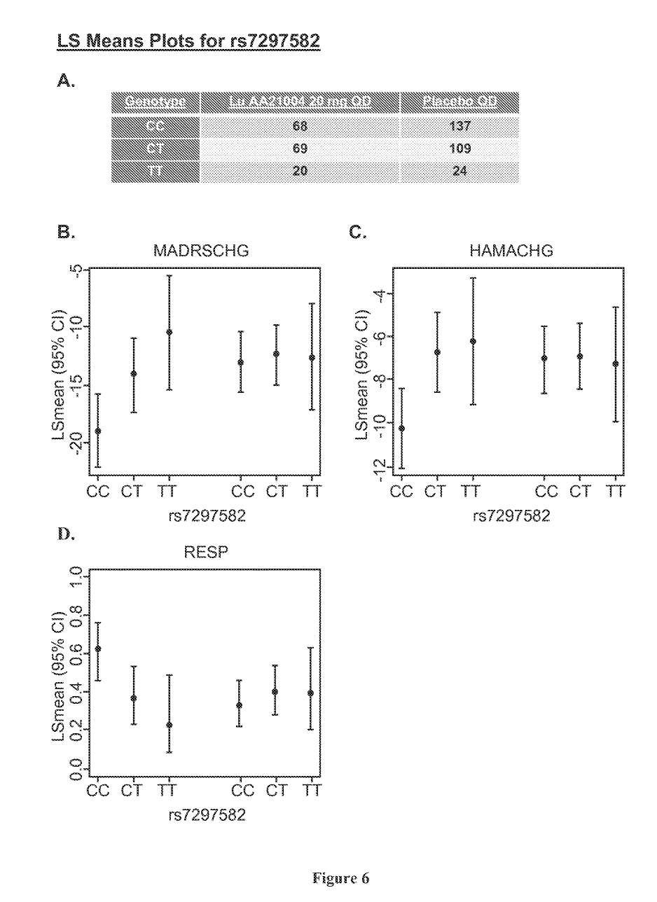

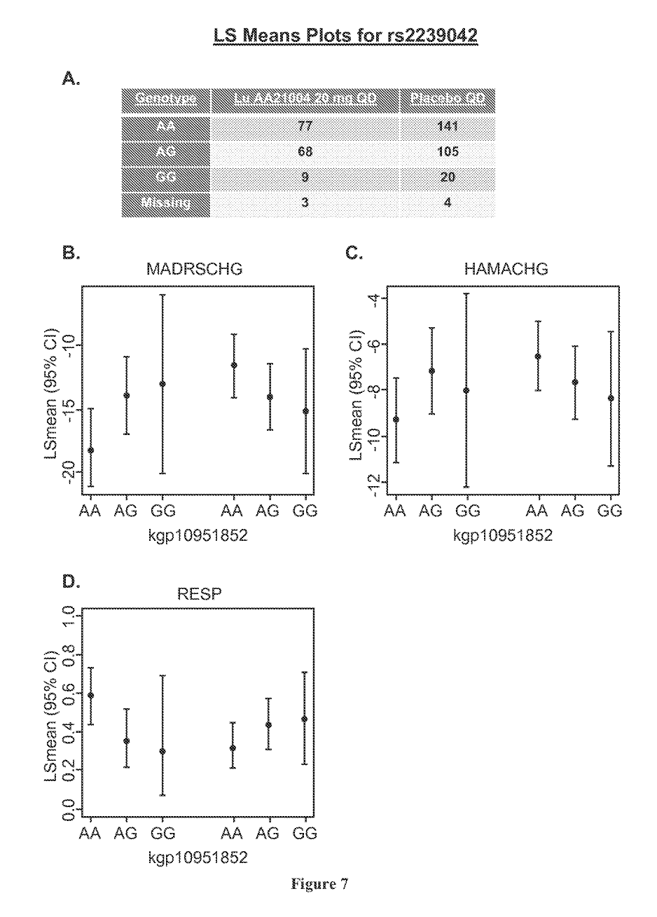

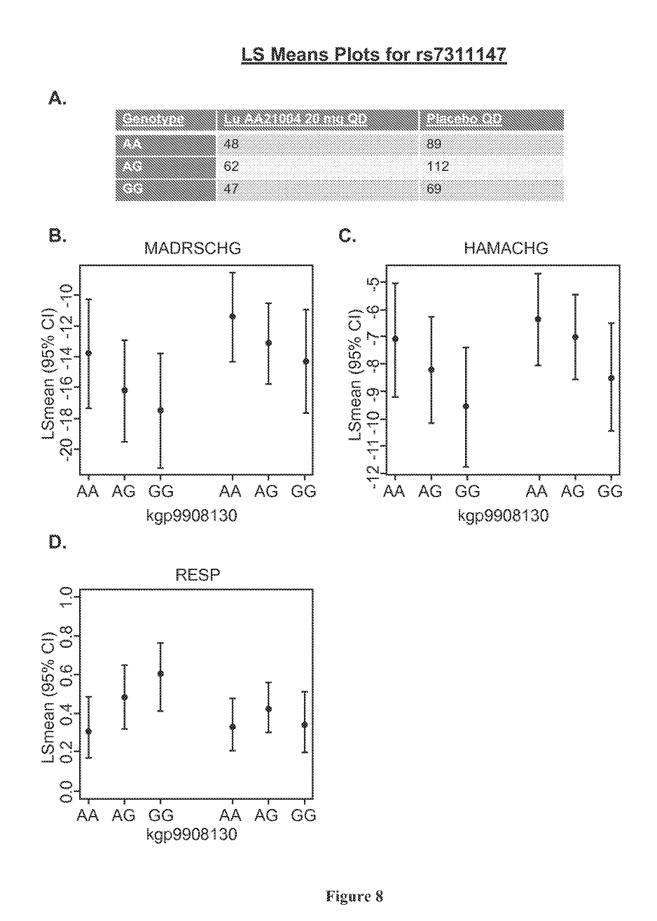

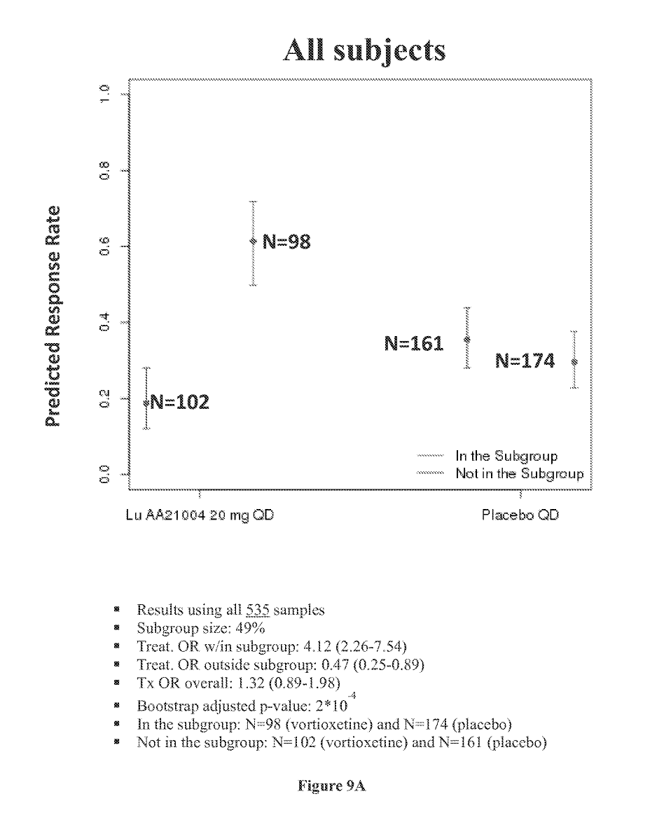

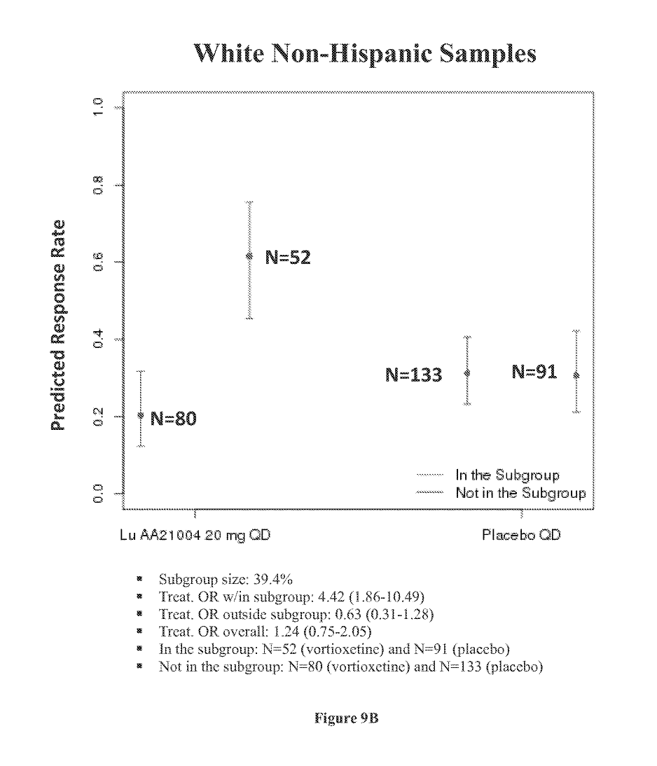

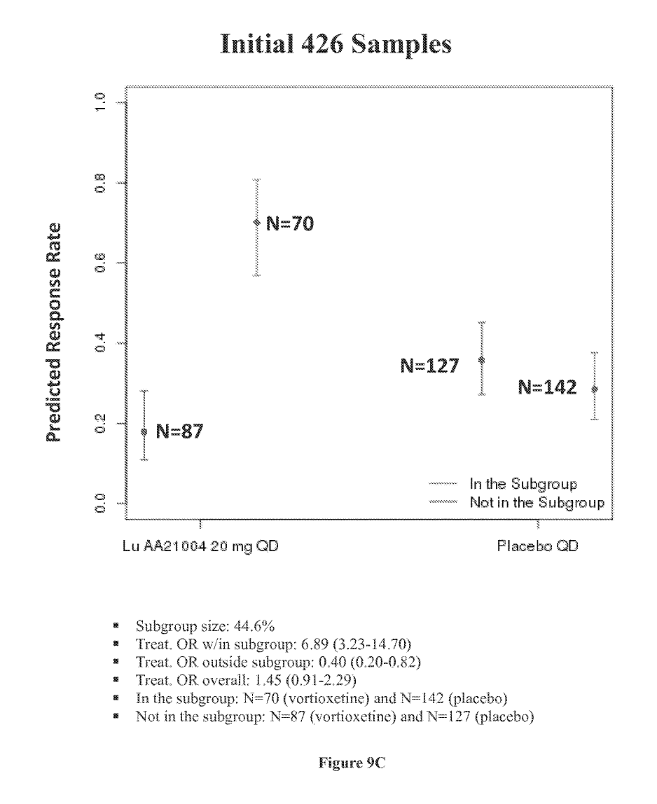

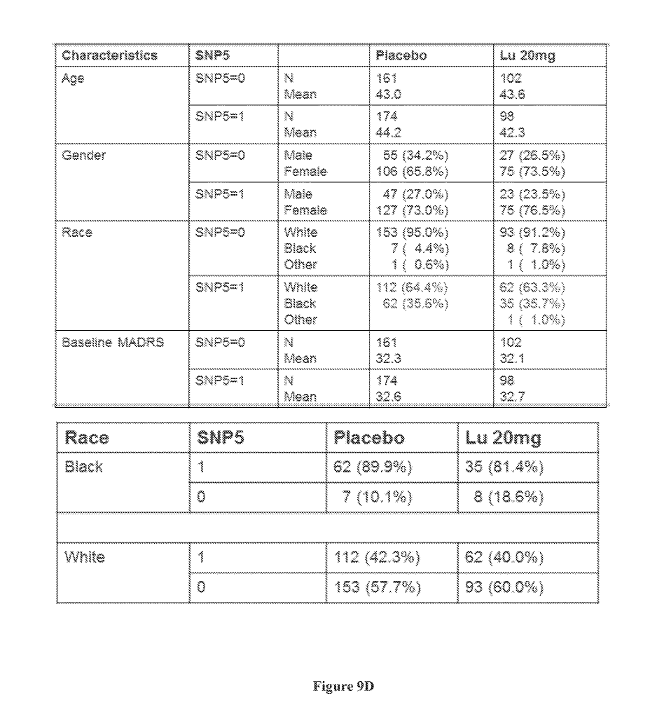

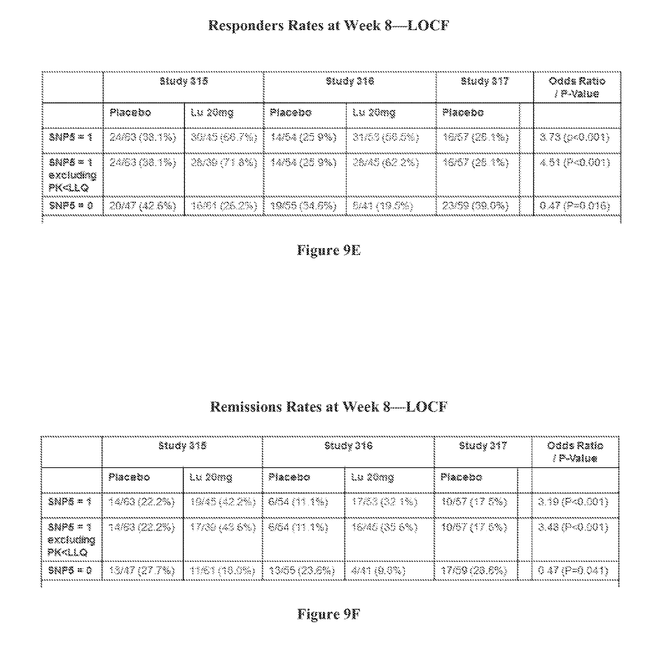

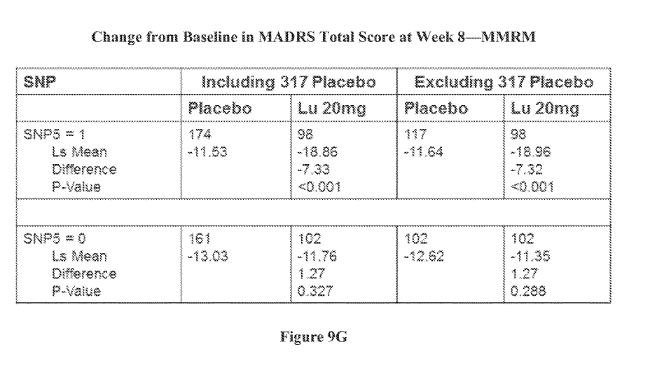

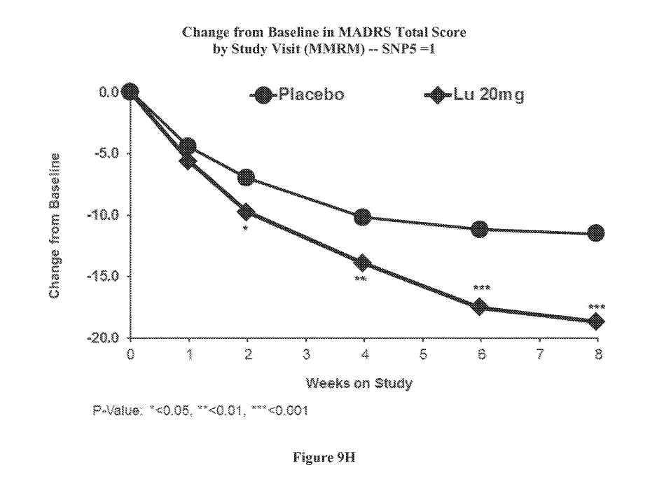

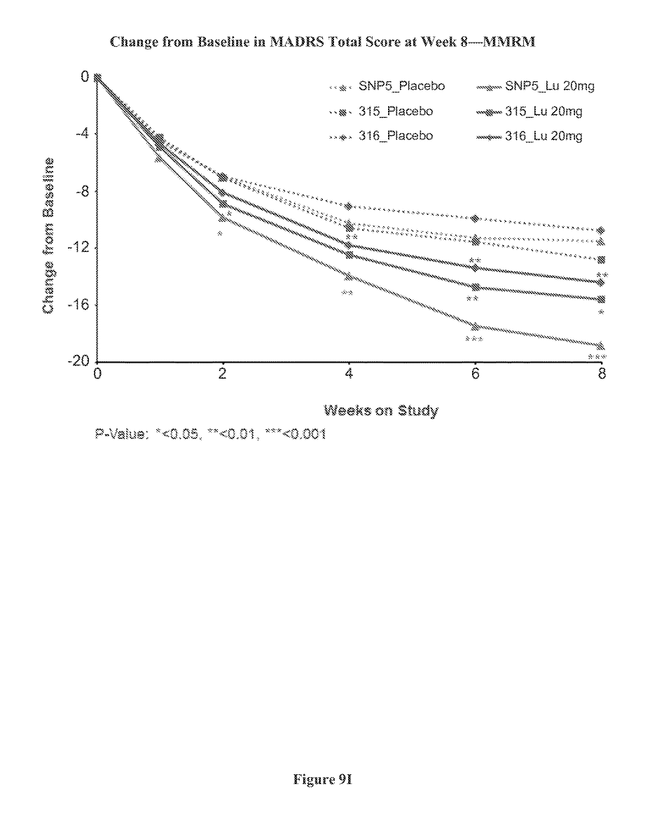

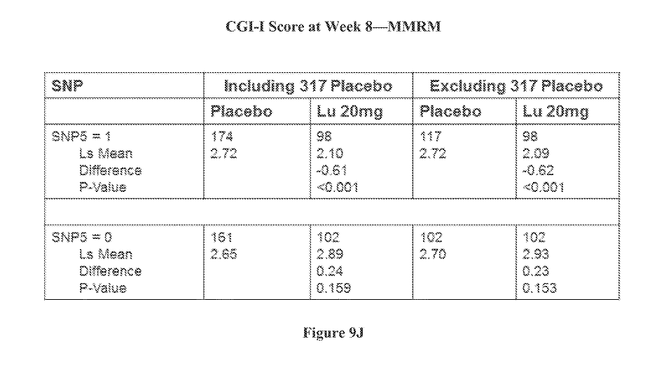

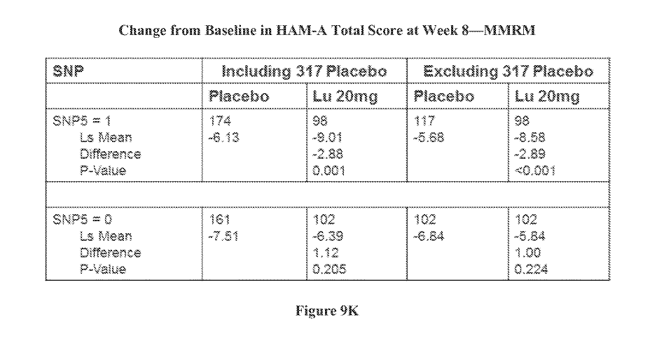

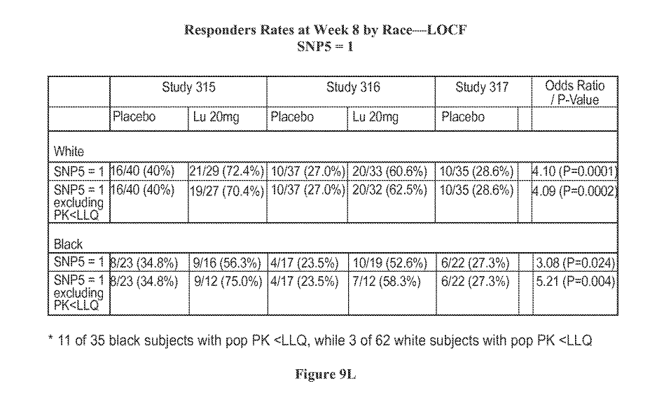

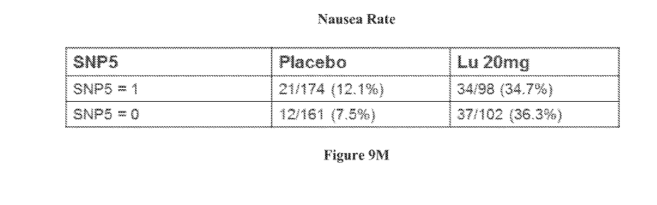

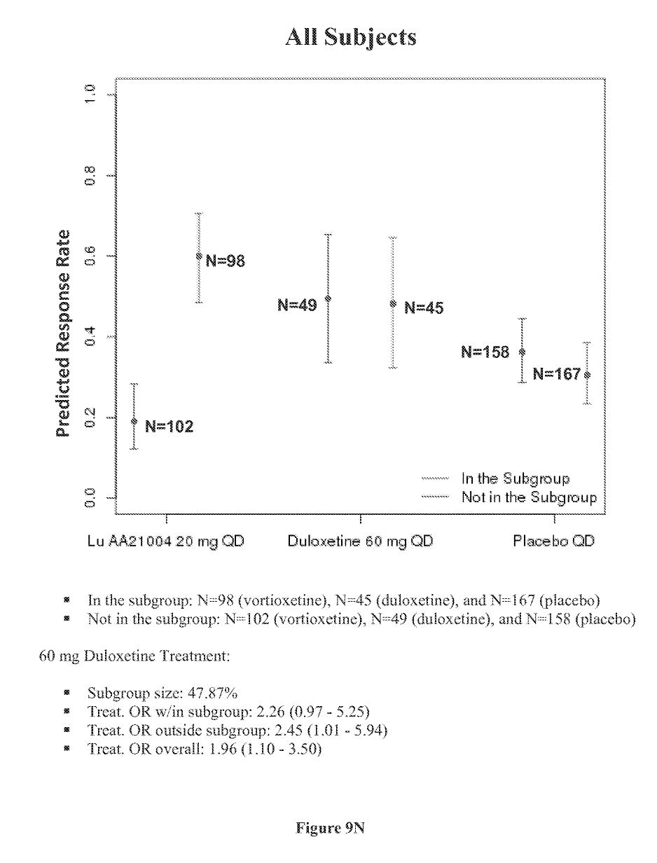

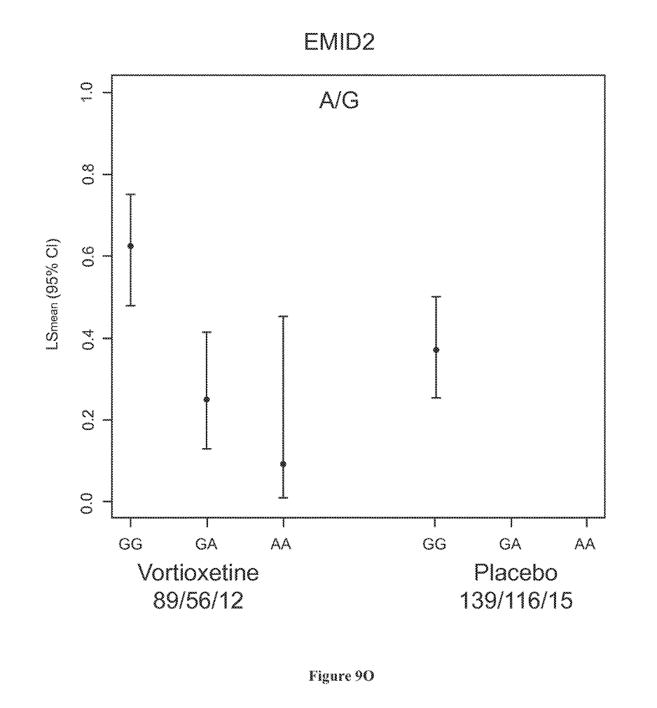

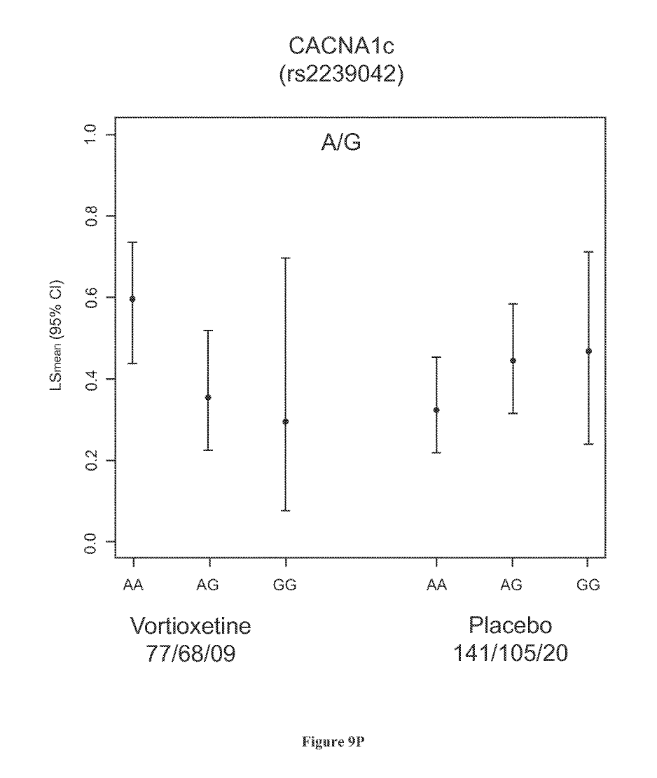

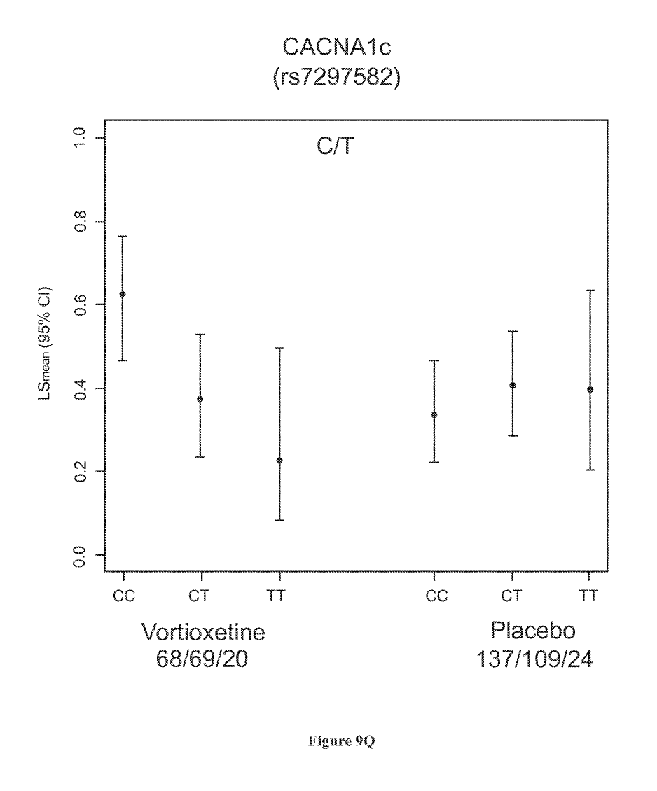

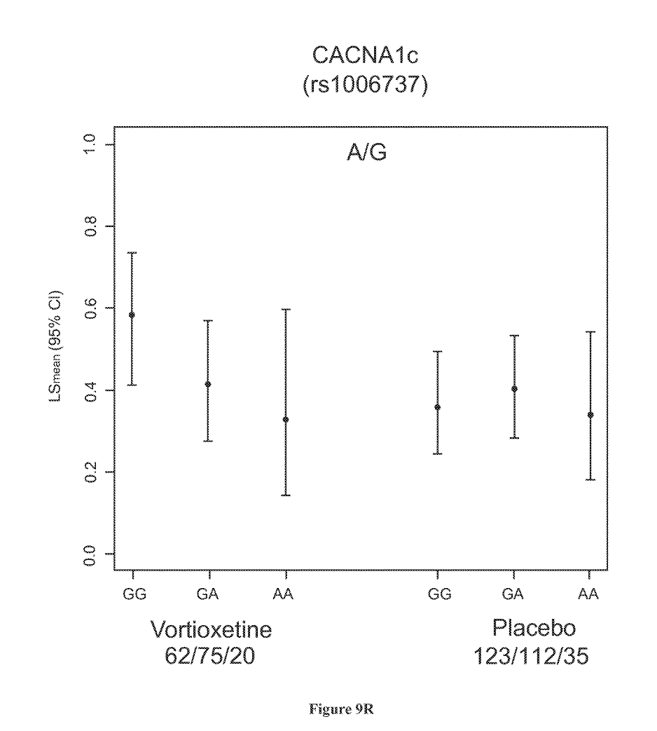

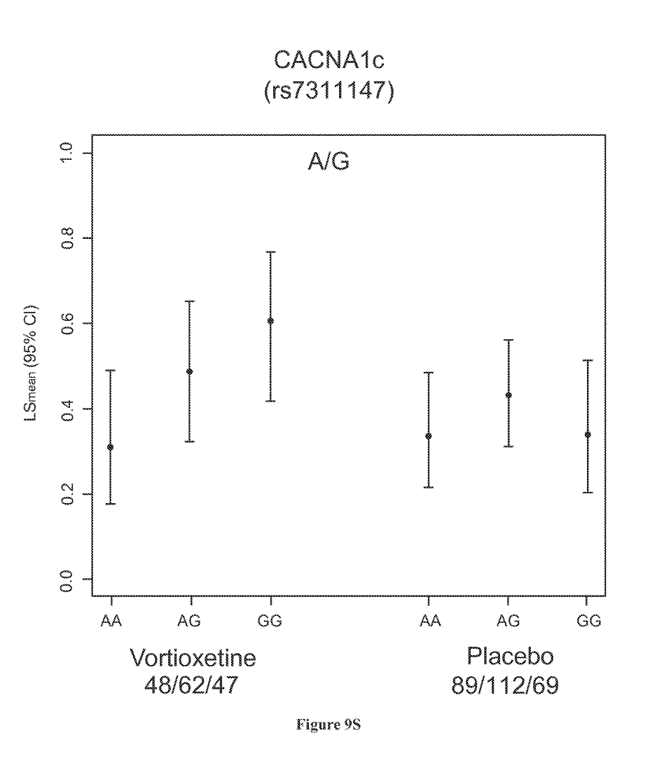

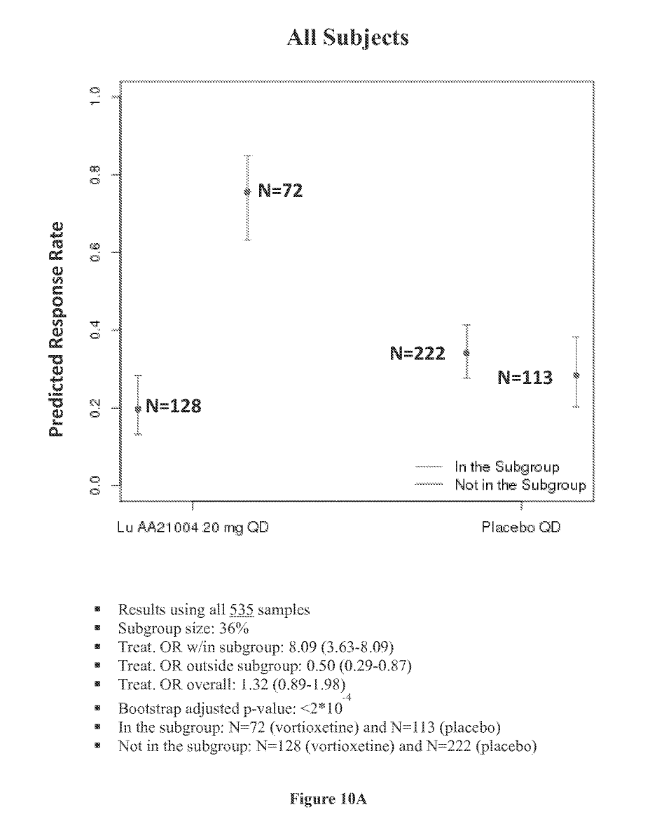

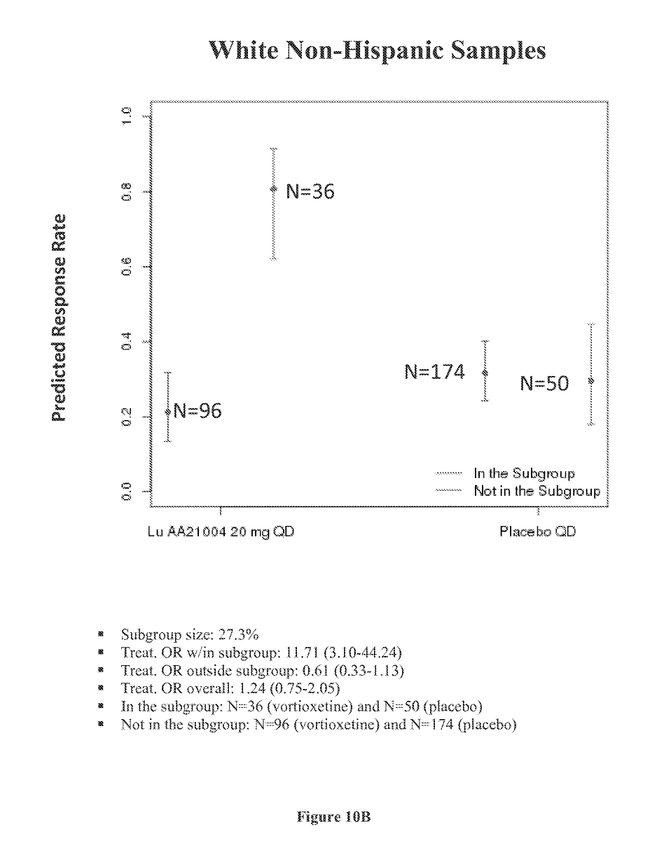

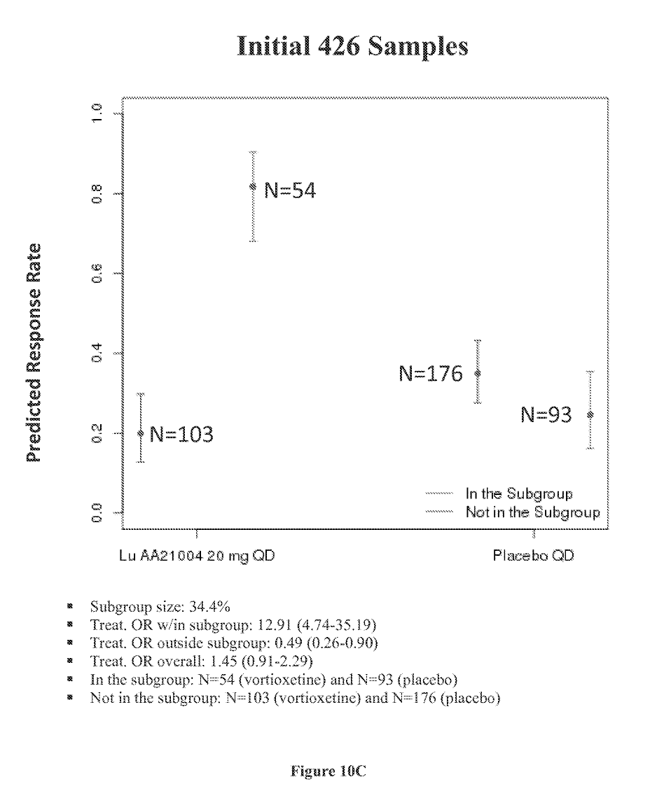

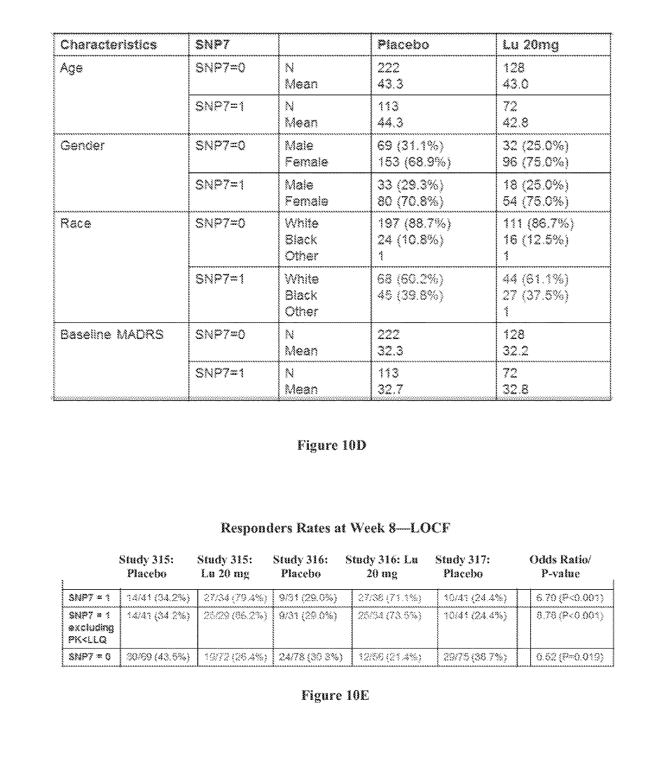

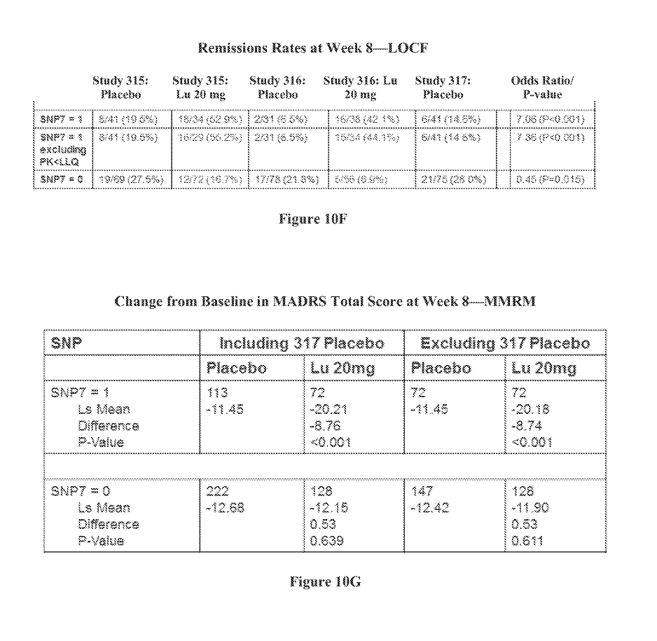

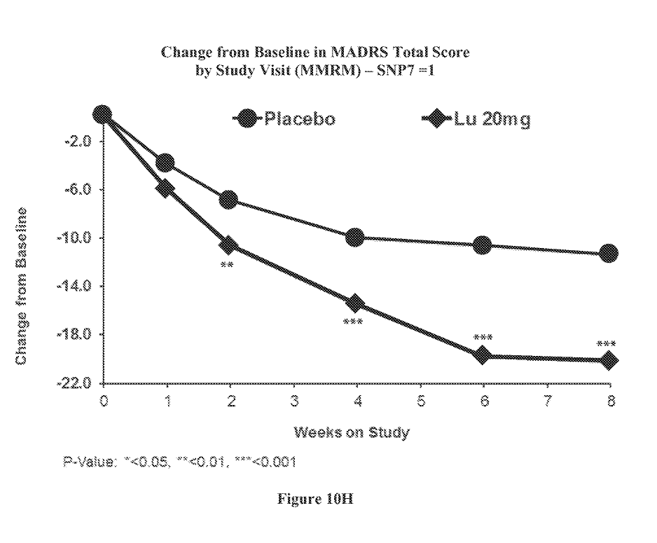

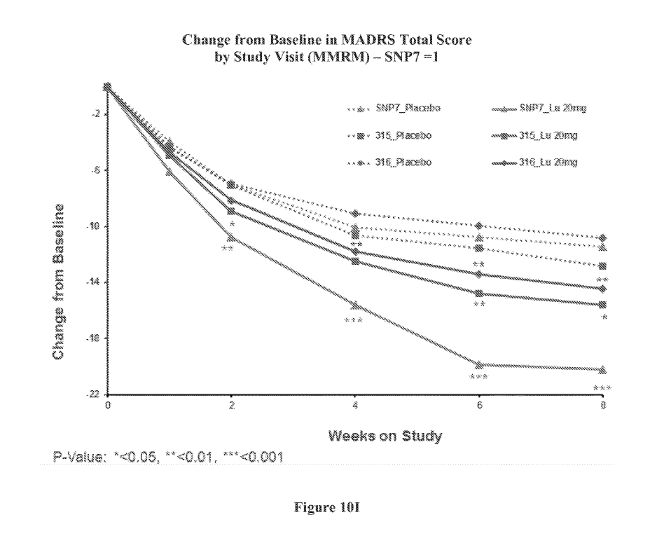

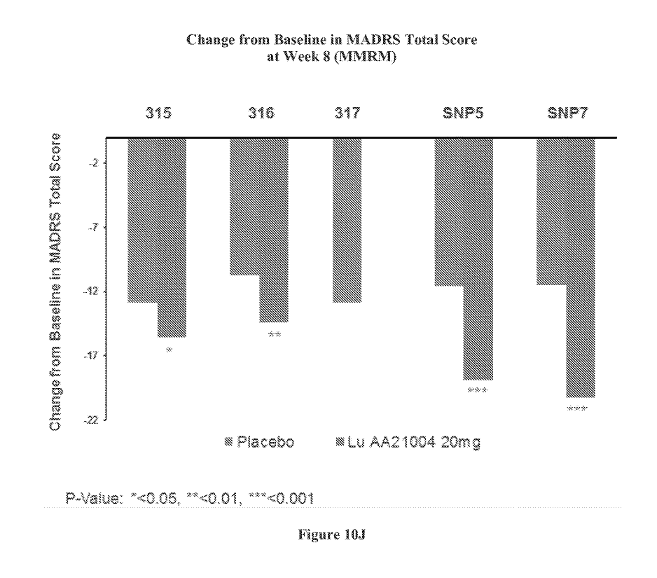

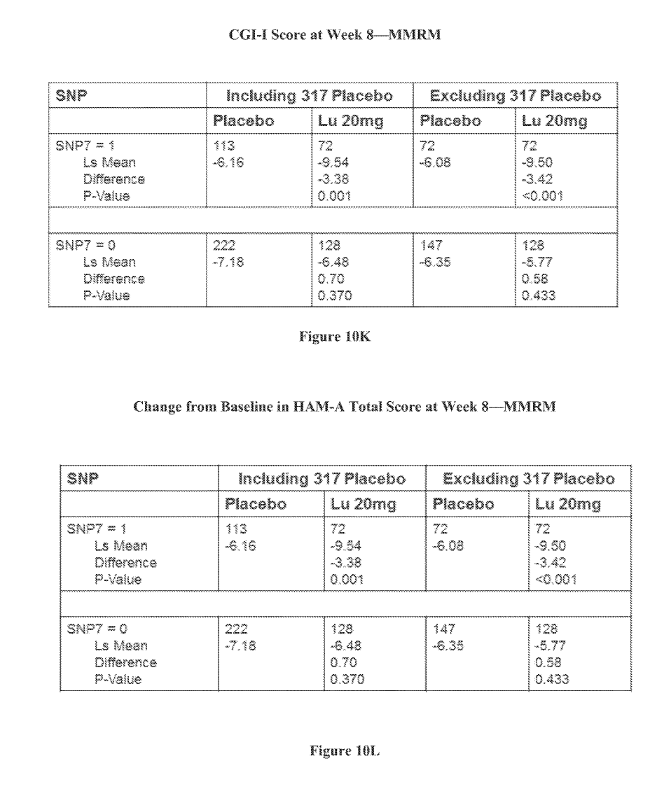

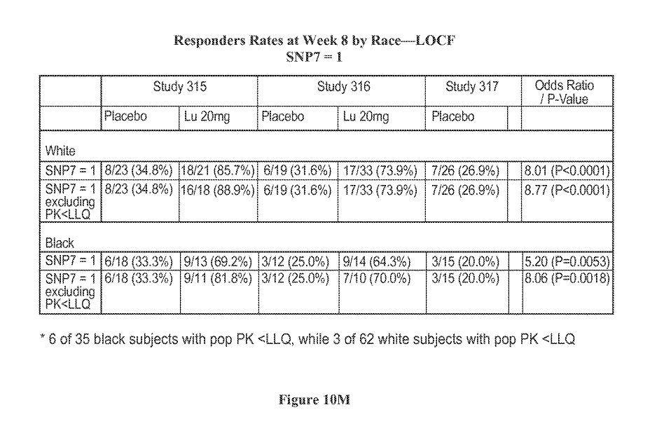

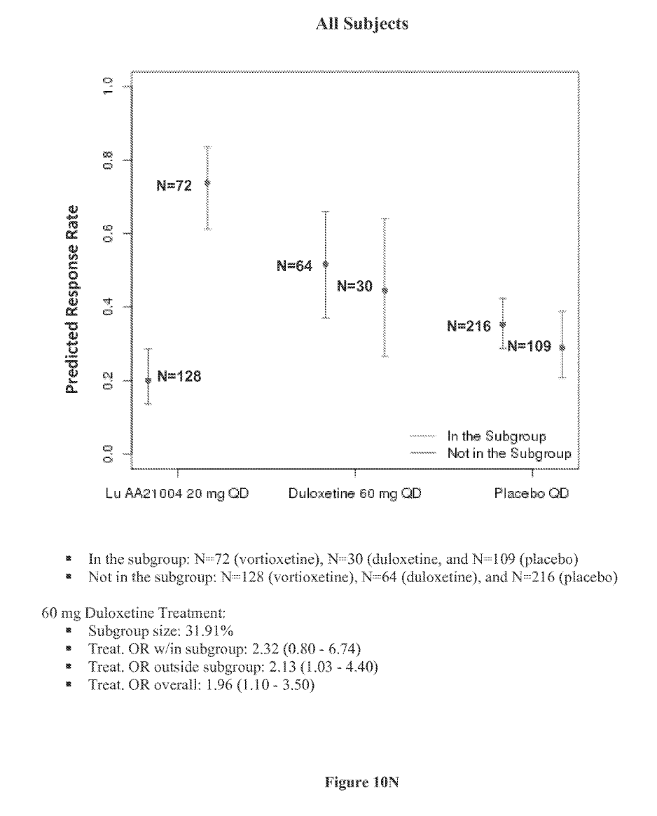

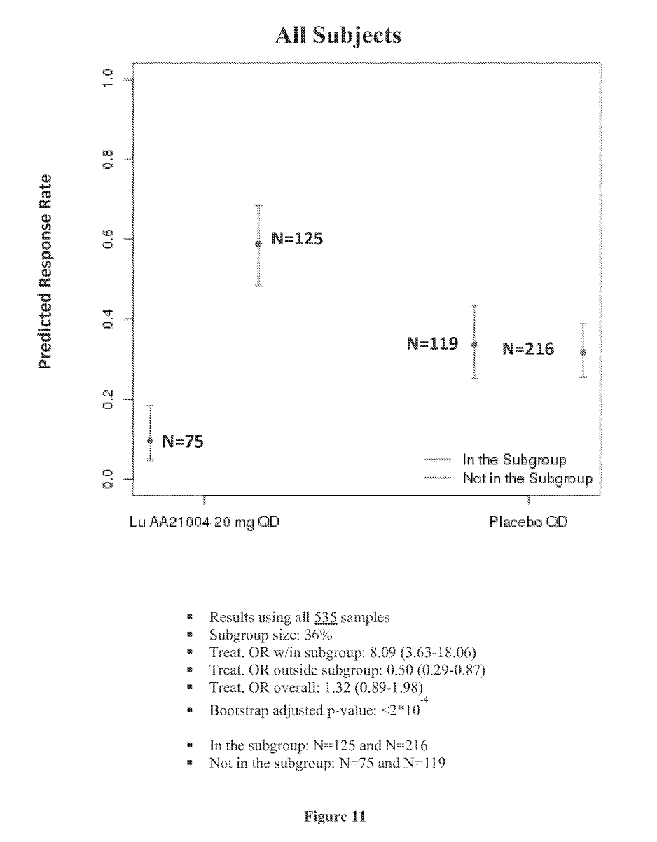

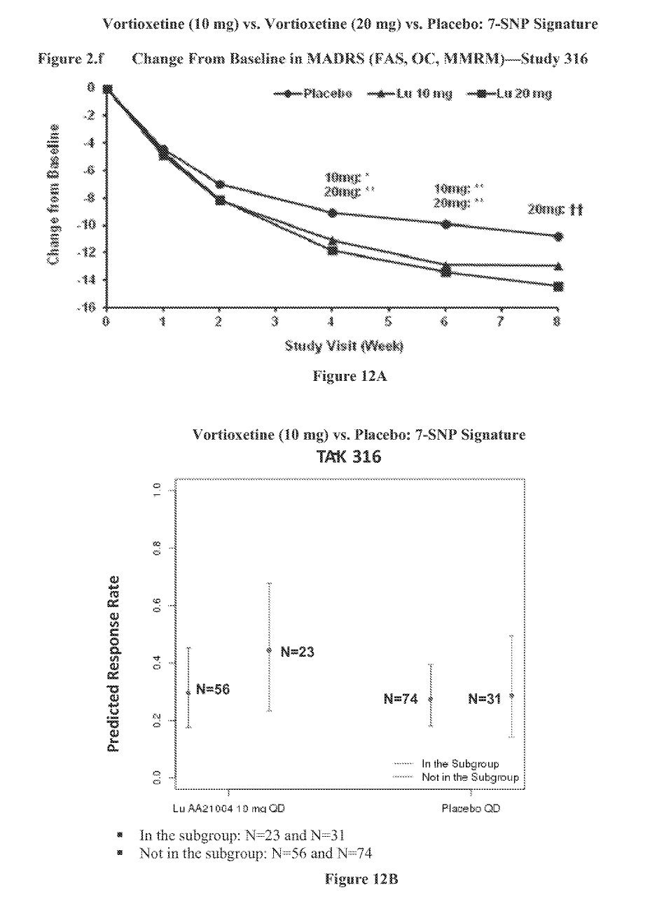

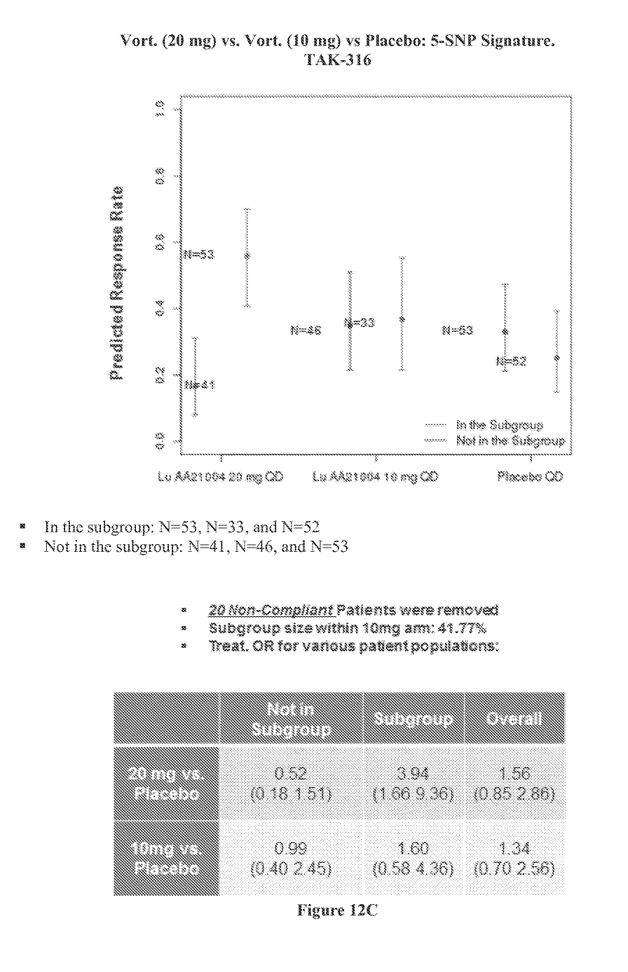

CACNA1C, COL26A1 rs4045, CSMD1, ZSCAN4, and ZNF551 showed statistically significant evidence for treatment specific effect. A subgroup identified via genetic signature based on CACNA1C+rs4045 showed statistically significant enhanced treatment effect. See FIGS. 2-4. A subgroup identified via genetic signature based on CACNA1C, COL26A1 rs4045, and CSMD1 also showed statistically significant enhanced treatment effect. See FIG. 9. A subgroup identified via genetic signature based on CACNA1C, COL26A1 rs4045, CSMD1, and ZSCAN4 also showed statistically significant enhanced treatment effect. See FIGS. 10 and 11.

### Example 2

The following tables demonstrate that individuals with COL26A1 rs4045 and a CACNA1C variant exhibited improved cognition when administered 20 mg/day of vortioxetine for 8 weeks.

TABLE 1
Response rate by treatment and by subgroup. Each element
is “# responders/# samples”

| | COL26A1 rs4045 and a | Variant | | |
|---|---|---|---|---|
| | CACNA1C variant positive | negative | N/A | Total |
| Lu 20 mg | 14/17 | 6/25 | 28/78 | 40/120 |
| Placebo | 5/17 | 6/30 | 21/81 | 48/128 |
| Total | 19/34 | 12/55 | 49/282 | 32/371 |

TABLE 2
Response by subgroup for all arms combined

| | No Response | Response |
|---|---|---|
| Not in Subgroup | 43 | 12 |
| In the Subgroup | 15 | 19 |
| <NA> | 193 | 89 |

TABLE 3
Response by subgroup for Lu 20 mg only

| | No Response | Response |
|---|---|---|
| Not in Subgroup | 19 | 6 |
| In the Subgroup | 3 | 14 |
| <NA> | 50 | 28 |

TABLE 4
Response by subgroup for Placebo only

| | No Response | Response |
|---|---|---|
| Not in Subgroup | 24 | 6 |
| In the Subgroup | 12 | 5 |
| <NA> | 60 | 21 |

### Example 3

Table 5 shows genes and gene combinations whose expression levels can be combined in multigene models that significantly correlate with overall response rate in adult MDD patients treated with 20 mg/day vortioxitene. In the table, the B allele represents the minor allele, and the A allele represents the major allele.

TABLE 5
5 variants used in genetic signature

| | | | | 95% CI | B | A | G = 0 | G = 1 | G = 2 |
|---|---|---|---|---|---|---|---|---|---|
| # | dbSNP ID | Gene | MAF | Coefficient β range | Allele | Allele | (AA) | (AB) | (BB) |
| 1 | rs4045 | EMID2 | 0.26 | −1.704 to −0.275 | A | G | GG | AG | AA |
| 2 | rs59420002 | CSMD1 | 0.04 | 0.484 to 6.017 | G | A | AA | GA | GG |
| 3 | rs7297582 | CACNA1C | 0.31 | −0.825 to 0.321 | T | C | CC | TC | TT |
| 4 | rs2239042 | CACNA1C | 0.27 | −1.529 to −0.150 | G | A | AA | GA | GG |
| 5 | rs7311147 | CACNA1C | 0.47 | −0.100 to 0.870 | G | A | AA | GA | GG |

MAF = Minor Allele Frequency

The 5-variant (5-SNP) model shows statistically significant evidence for a treatment-specific effect. A subgroup identified via the genetic signature set forth in Table 5 showed statistically significant enhanced treatment effect. See FIG. 9. Moreover, the subjects outside the subgroup showed a statistically significant non-response effect. See FIG. 9.

The subgroup showing enhanced treatment effect was identified using a combined elastic net/bootstrapping approach, similar to that set forth in Li et al., “A multi-marker molecular signature approach for treatment-specific subgroup identification with survival outcomes,” The Pharmacogenomics Journal, 14(5): 439-45 (2014), which is incorporated herein by reference and made a part hereof.

In particular, the dataset was bootstrapped 1000 times, and each bootstrapped dataset was used to re-estimate a score using elastic net. Using this approach, a coefficient range for each SNP was estimated at a 95% confidence interval (CI) using 2.5% and 97.5% percentiles of the 1000 estimates. The 95% CI coefficient ranges are set forth in Table 5. Using these coefficients, for each signature, the patient's score is calculated as:

$${Score}_{i} = {\sum }_{j} {G}_{j} {β}_{j}$$

where j indicates the jth SNP, and the patient's membership is

$${Membership}_{i} = \{ \begin{matrix}1 , if {Score}_{i} \geq threshold \\ 0 , if {Score}_{i} < threshold\end{matrix}$$

In the equation, G is 0, 1, or 2, depending on the patient's allele combination (see Table 5) and coefficient β is selected from within the range provided for each particular variant (also see Table 5). Using this equation, responders were identified using the following formula, where the optimal cutoff was τ*=−0.6:

Scoreᵢ=(rs4045coefficient)*rs4045+(rs59420002coefficient)*rs59420002+(rs7297582coefficient)*rs7297582+(rs2239042coefficient)*rs2239042+(rs7311147coefficient)*rs7311147

The specific algorithm used in this example was as follows:

Scoreᵢ=−0.9*rs4045+1.6*rs59420002−0.3*rs7297582−0.8*rs2239042+0.3*rs7311147

Demographics and baseline demographics and characteristics for subjects in the studies are shown in FIG. 9D for the 5-variant model, where SNP5=1 corresponds to patients in the subgroup identified by the 5-variant model and SNP5=0 corresponds to patients not in the subgroup.

From these studies, responder rates at week 8 (FIG. 9E) and remissions rates at week 8 (FIG. 9F) were determined via LOCF (last observation carried forward).

Also, change in baseline MADRS total scores at week 8 and at each visit were determined via MMRM (mixed model for repeated measurements) (FIGS. 9G-I). CGI-I scores (9J) and change from baseline in HAM-A total scores (9K) at week 8 were also calculated. Responder rates at week 8 based on race were calculated via LOCF and are shown in FIG. 9L.

Nausea rates we determined following 20 mg vortioxitene administration in the 5-variant model (FIG. 9M).

FIG. 9N compares treatment effect (i.e., odds ratio (OR)) with vortioxetine 20 mg QD, duloxetine 60 mg QD, and placebo QD in a 5-variant model. Enhanced treatment effect of vortioxetine relative to duloxetine is shown in Table 6, and treatment effect of duloxetine relative to placebo is shown in Table 7.

TABLE 6
Treatment with vortioxetine vs. duloxetine

| | Treat. OR (95% CL) |
|---|---|
| Not in SubGroup | 0.24 (0.07-0.86) |
| In SubGroup | 4.17 (1.15-16.67) |
| Overall | 0.82 (0.36-1.85) |

TABLE 7
Treatment with duloxetine vs. placebo

| | Treat. OR (95% CL) |
|---|---|
| Not in SubGroup | 2.29 (0.68-7.71) |
| In SubGroup | 1.54 (0.46-5.17) |
| Overall | 1.44(0.65-3.18) |

FIGS. 9O-S show overall response status plots for individuals with A/G EMID2 alleles (9O), A/G rs2239042 alleles (9P), C/T rs7297582 alleles (9Q), A/G rs1006737 alleles (9R), and A/G rs7311147 alleles (9S).

### Example 4

Table 8 shows genes and gene combinations whose expression levels can be combined in multigene models that significantly correlate with overall response rate in adult MDD patients treated with 20 mg/day vortioxitene. In the table, the B allele represents the minor allele, and the A allele represents the major allele.

TABLE 8
7 variants used in genetic signature

| | | | | 95% CI | B | A | G = 0 | G = 1 | G = 2 |
|---|---|---|---|---|---|---|---|---|---|
| # | dbSNP ID | Gene | MAF | Coefficient β range | Allele | Allele | (AA) | (AB) | (BB) |
| 1 | rs4045 | EMID2 | 0.26 | −1.614 to −0.221 | A | G | GG | AG | AA |
| 2 | rs59420002 | CSMD1 | 0.04 | 0.276 to 5.107 | G | A | AA | GA | GG |
| 3 | rs7297582 | CACNA1C | 0.31 | −0.698 to 0.422 | T | C | CC | TC | TT |
| 4 | rs2239042 | CACNA1C | 0.27 | −1.375 to −0.014 | G | A | AA | GA | GG |
| 5 | rs7311147 | CACNA1C | 0.47 | −0.011 to 0.929 | G | A | AA | GA | GG |
| 6 | rs12983596 | ZSCAN4 | 0.37 | −1.524 to 0.000 | C | T | TT | CT | CC |
| 7 | rs9749513 | ZSCAN4 | 0.45 | −1.101 to 0.384 | C | T | TT | CT | CC |

MAF = Minor Allele Frequency

The 7-variant (7-SNP) model shows statistically significant evidence for a treatment-specific effect. A subgroup identified via the genetic signature set forth in Table 8 showed statistically significant enhanced treatment effect. See FIG. 10. Moreover, the subjects outside the subgroup showed a statistically significant non-response effect. See FIG. 10.

The subgroup showing enhanced treatment effect was identified using the elastic net/bootstrapping methods set forth in Example 3. In particular, responders were identified using the following formula, where the optimal cutoff was τ*=−0.6:

Scoreᵢ=(rs4045coefficient)*rs4045+(rs59420002coefficient)*rs59420002+(rs7297582coefficient)*rs7297582+(rs2239042coefficient)*rs2239042+(rs7311147coefficient)*rs7311147+(rs12983596coefficient)*rs12983596+(rs9749513coefficient)*rs9749513

The specific algorithm used in this example was as follows:

Scoreᵢ=−0.8*rs4045+1.3*rs59420002−0.1*rs7297582−0.6*rs2239042+0.4*rs7311147−0.5*rs12983596−0.4*rs9749513

Demographics and baseline demographics and characteristics for subjects in the studies are shown in FIG. 10D for the 7-variant model, where SNP7=1 corresponds to patients in the subgroup identified by the 7-variant model and SNP7=0 corresponds to patients not in the subgroup.

From these studies, responder rates at week 8 (FIG. 10E) and remissions rates at week 8 (FIG. 10F) were determined via LOCF.

Also, change in baseline MADRS total scores at week 8 and at each visit were determined via MMRM (FIGS. 10G-I). A comparison of the change from baseline in MADRS total score in the 5-variant model and 7-variant model is shown in FIG. 10J.

CGI-I scores (10K) and change from baseline in HAM-A total scores (10L) at week 8 were also calculated. Responder rates at week 8 based on race were calculated via LOCF and are shown in FIG. 10M.

FIG. 10N compares treatment effect with vortioxetine 20 mg QD, duloxetine 60 mg QD, and placebo QD. A comparison of enhanced treatment effects is shown in Tables 9 and 10.

TABLE 9
Treatment with vortioxetine vs. duloxetine

| | Treat. OR (95% CL) |
|---|---|
| Not in SubGroup | 0.30 (0.09-0.98) |
| In SubGroup | 6.25 (1.41-33.33) |
| Overall | 0.82 (0.36-1.85) |

TABLE 10
Treatment with duloxetine vs. placebo

| | Treat. OR (95% CL) |
|---|---|
| Not in SubGroup | 1.51 (0.51-4.42) |
| In SubGroup | 1.87 (0.49-7.15) |
| Overall | 1.44(0.65-3.18) |

### Example 5

Table 11 shows genes and gene combinations whose expression levels can be combined in multigene models that significantly correlate with overall response rate in adult MDD patients treated with 20 mg/day vortioxitene. In the table, the B allele represents the minor allele, and the A allele represents the major allele.

TABLE 11
14 variants used in genetic signature

| | | | 95% CI | B | A | G = 0 | G = 1 | G = 2 |
|---|---|---|---|---|---|---|---|---|
| # | dbSNP ID | Gene | Coefficient β range | Allele | Allele | (AA) | (AB) | (BB) |
| 1 | rs4045 | EMID2 | −1.896 to −0.278 | A | G | GG | AG | AA |
| 2 | rs59420002 | CSMD1 | 0.326 to 4.771 | G | A | AA | GA | GG |
| 3 | rs7297582 | CACNA1C | −0.825 to 0.467 | T | C | CC | TC | TT |
| 4 | rs2239042 | CACNA1C | −1.621 to −0.120 | G | A | AA | GA | GG |
| 5 | rs7311147 | CACNA1C | −0.116 to 0.980 | G | A | AA | GA | GG |
| 6 | rs9304796 | ZSCAN4 | −0.852 to 0.171 | T | G | GG | TG | TT |
| 7 | rs73064580 | ZSCAN4 | −0.033 to 0.605 | C | T | TT | CT | CC |
| 8 | rs12983596 | ZSCAN4 | −2.788 to 0.139 | C | T | TT | CT | CC |
| 9 | rs12984275 | ZSCAN4 | −0.795 to 0.151 | G | C | CC | GC | GG |
| 10 | rs9749513 | ZSCAN4 | −1.270 to 0.910 | C | T | TT | CT | CC |
| 11 | rs12609579 | ZSCAN4 | −0.026 to 0.569 | A | C | CC | AC | AA |
| 12 | rs4239480 | ZSCAN4 | −0.021 to 0.548 | A | G | GG | AG | AA |
| 13 | rs9676604 | ZSCAN4 | −2.081 to 0.854 | T | C | CC | TC | TT |
| 14 | rs12162232 | ZSCAN4 | −0.673 to 0.172 | A | G | GG | AG | AA |

The 14-variant model shows statistically significant evidence for a treatment-specific effect. A subgroup identified via the genetic signature set forth in Table 11 showed statistically significant enhanced treatment effect. See FIG. 11. Moreover, the subjects outside the subgroup showed a statistically significant non-response effect. See FIG. 11.

The subgroup showing enhanced treatment effect was identified using the elastic net/bootstrapping methods set forth in Example 3. In particular, responders were identified using the following formula, where the optimal cutoff was τ*=−1.4:

Scoreᵢ=(rs4045coefficient)*rs4045+(rs59420002coefficient)*rs59420002+(rs7297582coefficient)*rs7297582+(rs2239042coefficient)*rs2239042+(rs7311147coefficient)*rs7311147+(rs9304796coefficient)*rs9304796+(rs73064580coefficient)*rs73064580+(rs12983596coefficient)*rs12983596+(rs12984275coefficient)*rs12984275+(rs9749513coefficient)*rs9749513+(rs12609579coefficient)*rs12609579+(rs4239480coefficient)*rs4239480+(rs9676604coefficient)*rs9676604+(rs12162232coefficient)*rs12162232

The specific algorithm used in this example was as follows:

Scoreᵢ=−0.8*rs4045+1.2*rs59420002−0.1*rs7297582−0.7*rs2239042+0.3*rs7311147−0.1*rs9304796+0.1*rs73064580−0.4*rs12983596−0.1*rs12984275−0.3*rs9749513+0.1*rs12609579+0.1*rs4239480+0.1*rs9676604−0.05*rs12162232

### Example 6

Table 12 shows genes and gene combinations whose expression levels can be used in multigene models that significantly correlate with overall response rate in adult MDD patients treated with 20 mg/day vortioxitene. The SNPs listed in Table 12 were shown to be interchangeable with SNPs in the 7-gene and 14-gene models, shown above, without any significant change in treatment response effect or non-response effect.

TABLE 12
SNPs on chromosome 19: ZSCAN4 and ZNF551

| | | | | Major | Minor |
|---|---|---|---|---|---|
| Chromosome | dbSNP ID | Gene | Position | Allele | Allele |
| 19 | rs9304796 | ZSCAN4 | 58177590 | G | T |
| 19 | rs73064580 | ZSCAN4 | 58178398 | T | C |
| 19 | rs12983596 | ZSCAN4 | 58178505 | T | C |
| 19 | rs12984275 | ZSCAN4 | 58181845 | C | G |
| 19 | rs9749513 | ZSCAN4 | 58183476 | T | C |
| 19 | rs12609579 | ZSCAN4 | 58183668 | C | A |
| 19 | rs4239480 | ZSCAN4 | 58184340 | G | A |
| 19 | rs9676604 | ZSCAN4 | 58189287 | C | T |
| 19 | rs12162232 | ZSCAN4 | 58194405 | G | A |
| 19 | rs10417057 | ZSCAN4 | 58177308 | T | C |
| 19 | rs10403851 | ZSCAN4 | 58179234 | G | A |
| 19 | rs56066537 | ZSCAN4 | 58181102 | G | T |
| 19 | rs112783430 | ZSCAN4 | 58185117 | G | T |
| 19 | rs9749360 | ZSCAN4 | 58186051 | A | G |
| 19 | rs12162230 | ZNF551 | 58194388 | G | A |

### Example 7

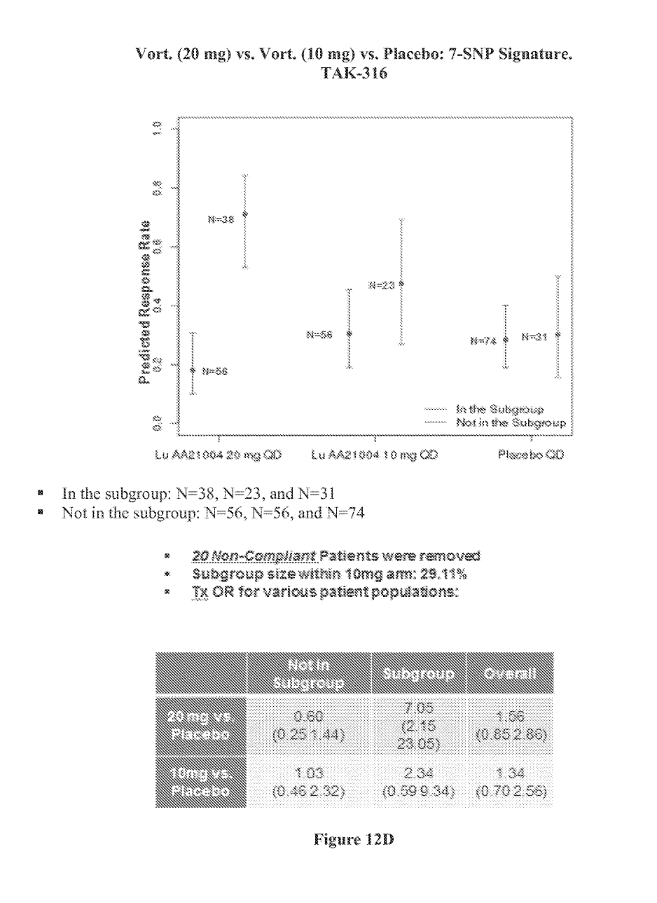

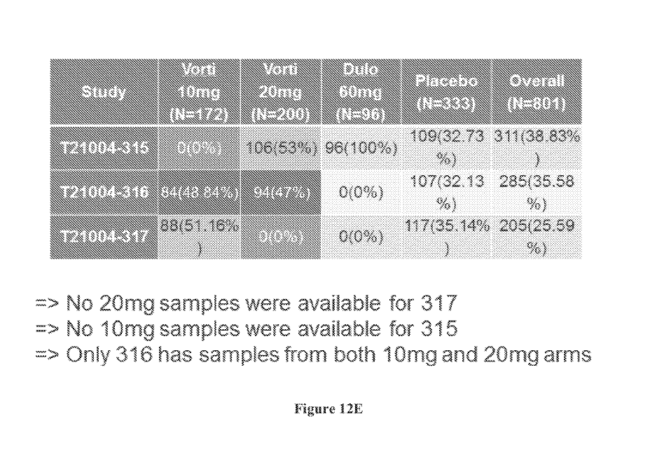

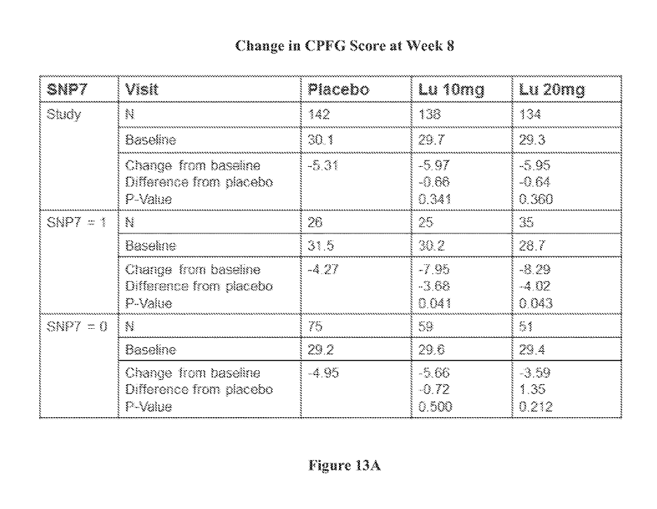

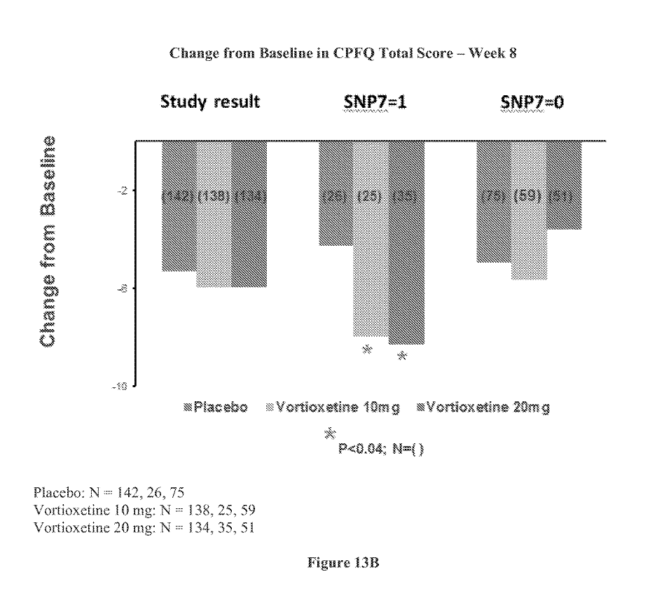

The 5-variant and 7-variant models discussed above show evidence for a treatment-specific effect with both 10 mg vortioxetine and 20 mg vortioxetine. This effect is demonstrated by MADRS scores obtained during treatment with 20 mg vortioxetine and 10 mg vortioxetine. See FIG. 12A, which shows MADRS scores obtained in the 7-SNP model. A least square means plot of MADRS scores obtained during treatment with 10 mg vortioxetine is shown in FIG. 12B using the 7-variant model. A comparison of the treatment effect of 20 mg vortioxetine, 10 mg vortioxetine, and a placebo using a 5-variant model is shown in FIG. 12C and using a comparison of the treatment effect in a 7-variant model is shown in FIG. 12D.

FIG. 12E shows sample accountability for 3 separate studies referred to in this example after removal of data corresponding to 20 non-compliant patients.

### Example 8

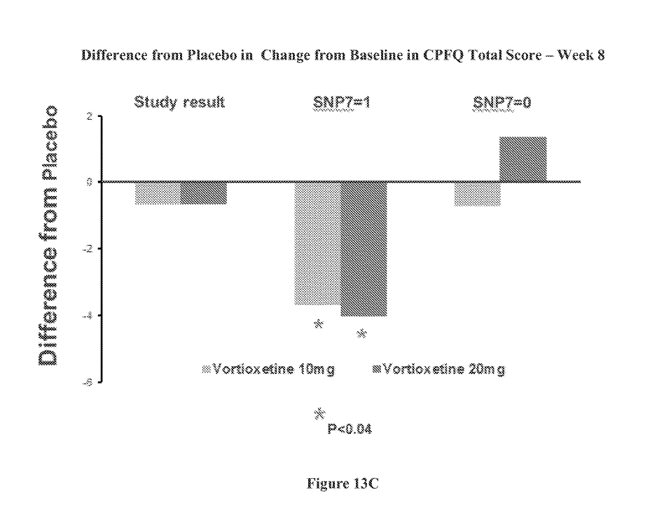

The 7-variant model discussed above in Example 4 showed evidence for improved cognition in adult MDD patients following administration of both 10 mg/day and 20 mg/day vortioxetine. This effect is demonstrated in Cognitive and Physical Functioning Questionnaire (CPFQ) results shown in FIG. 13A. This data is graphically represented as a change from baseline in the CPFQ total score in FIG. 13B. FIG. 13C shows a graphical representation of the 10 mg/day and 20 mg/day vortioxetine data relative to placebo data. In FIG. 13, the term “SNP7=1” corresponds to patients in the subgroup identified by the 7-variant model; the term “SNP7=0” corresponds to patients not in the subgroup.

### Example 9

Table 13 shows genes and gene combinations whose expression levels can be combined in multigene models that significantly correlate with overall response rate in adult MDD patients treated with 20 mg/day vortioxitene. In the table, the B allele represents the minor allele, and the A allele represents the major allele.

TABLE 13
10 variants used in genetic signature

| | | | 95% CI | B | A | G = 0 | G = 1 | G = 2 |
|---|---|---|---|---|---|---|---|---|
| # | dbSNP ID | Gene | Coefficient β range | Allele | Allele | (AA) | (AB) | (BB) |
| 1 | rs4045 | EMID2 | −1.846 to −0.299 | A | G | GG | AG | AA |
| 2 | rs59420002 | CSMD1 | 0.341 to 4.004 | G | A | AA | GA | GG |
| 3 | rs7297582 | CACNA1C | −0.982 to 0.114 | T | C | CC | TC | TT |
| 4 | rs2239042 | CACNA1C | −1.516 to −0.121 | G | A | AA | GA | GG |
| 5 | rs7311147 | CACNA1C | 0.000 to 1.093 | G | A | AA | GA | GG |
| 6 | rs12983596 | ZSCAN4 | −1.440 to 0.000 | C | T | TT | CT | CC |
| 7 | rs9749513 | ZSCAN4 | −1.258 to 0.000 | C | T | TT | CT | CC |
| 8 | rs62104612 | DYM | 0.137 to 1.420 | A | G | GG | AG | AA |
| 9 | rs1998609 | Intergenic | −1.741 to −0.409 | C | T | TT | CT | CC |
| 10 | rs4142192 | Intergenic | 0.000 to 1.257 | C | T | TT | CT | CC |

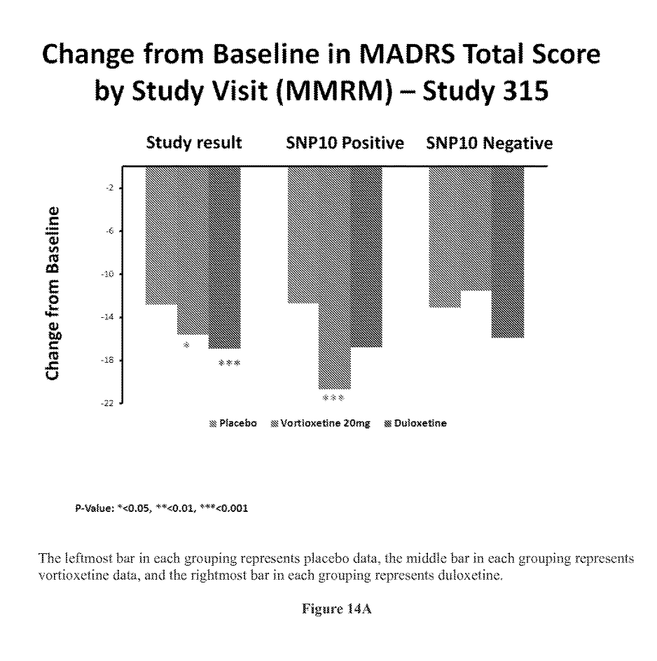

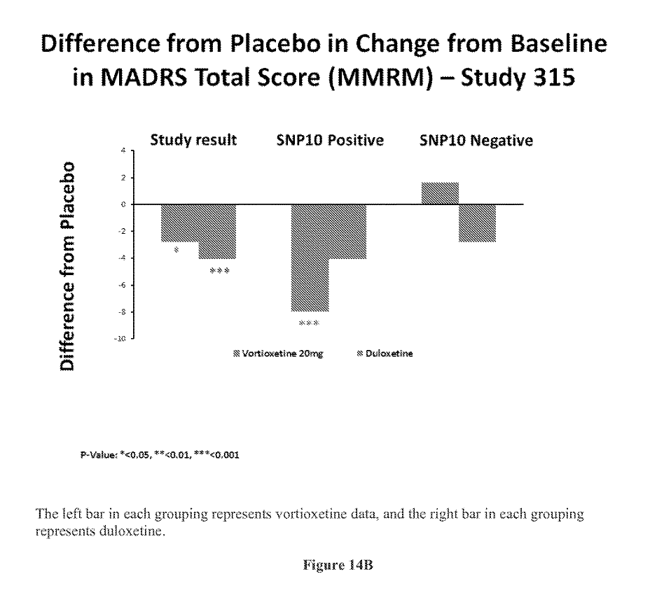

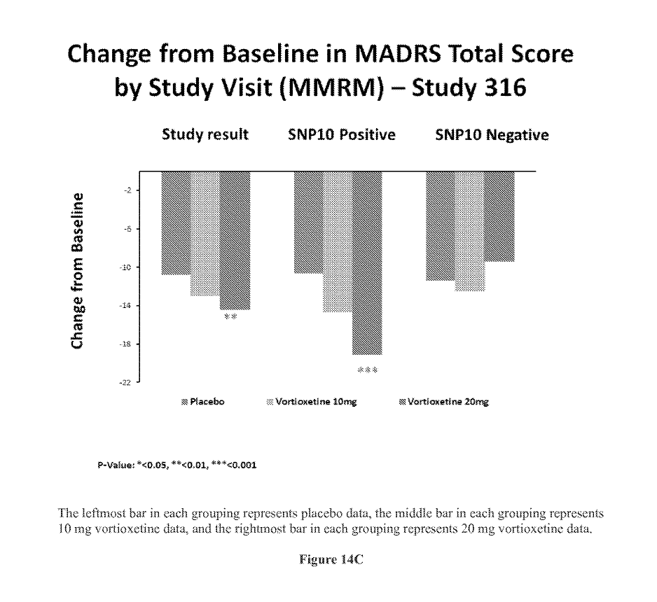

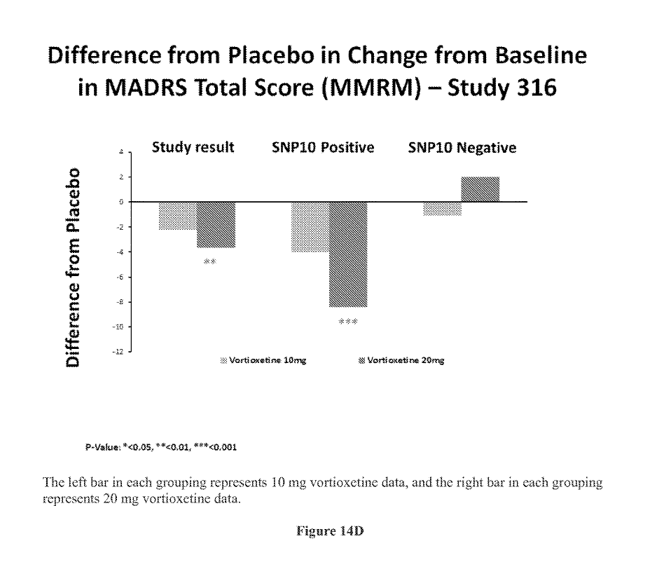

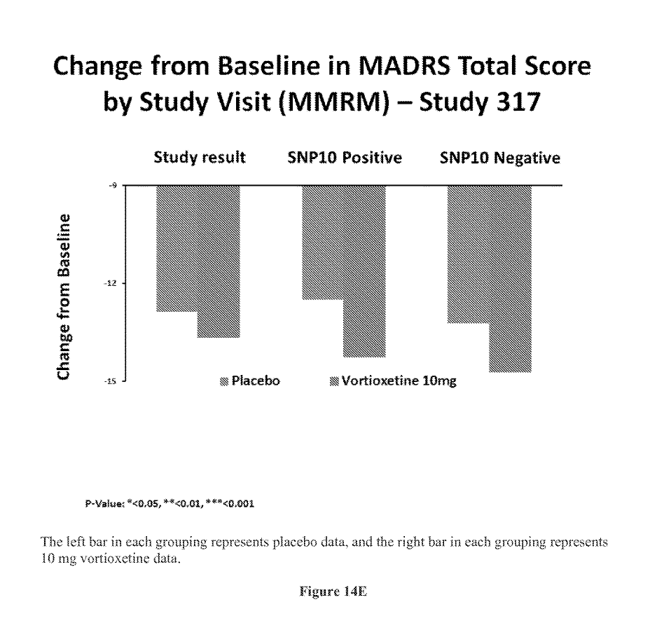

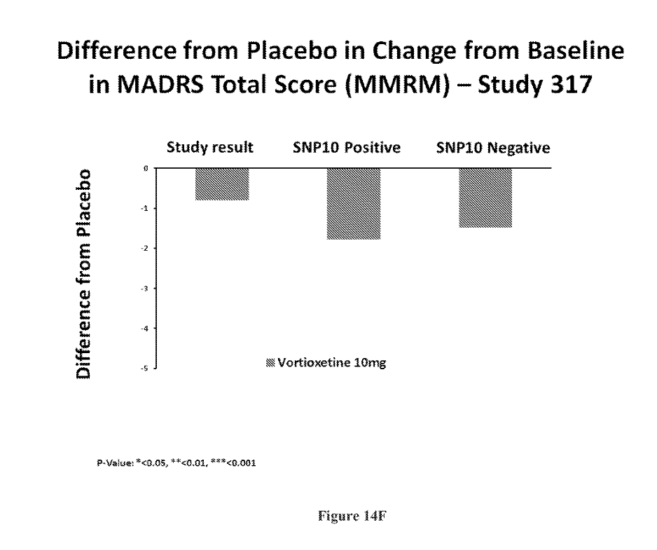

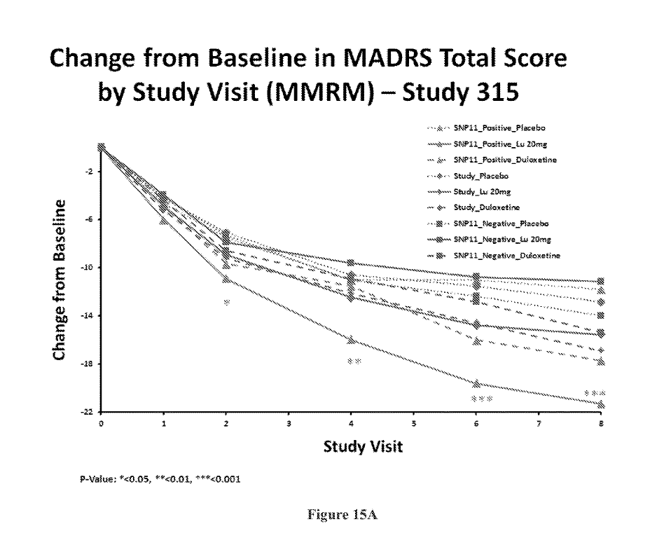

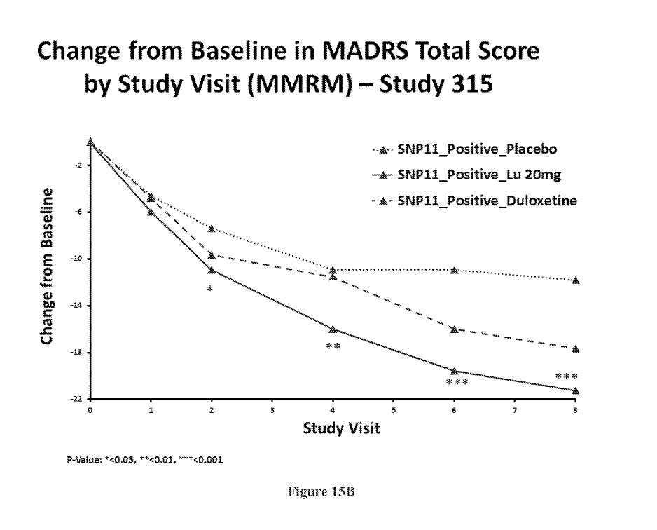

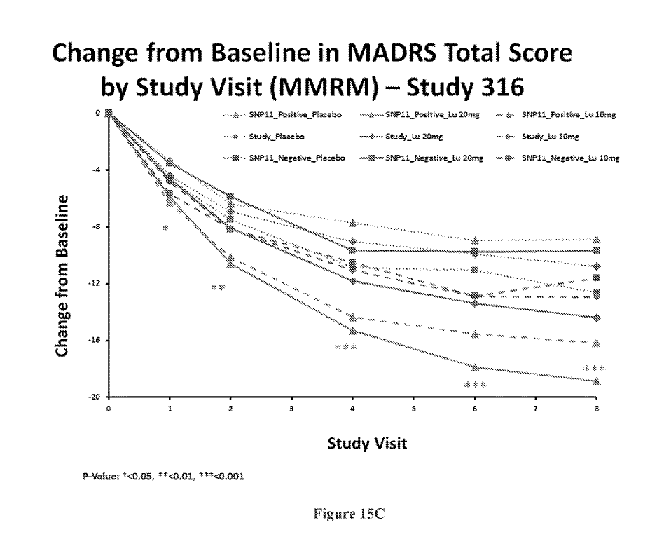

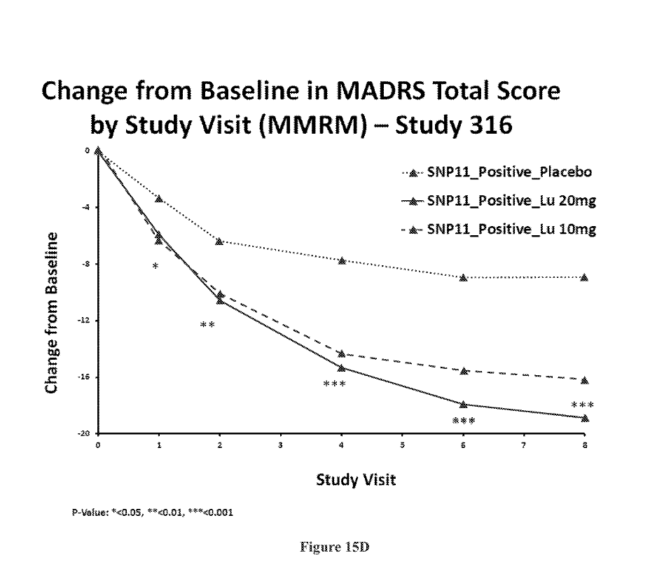

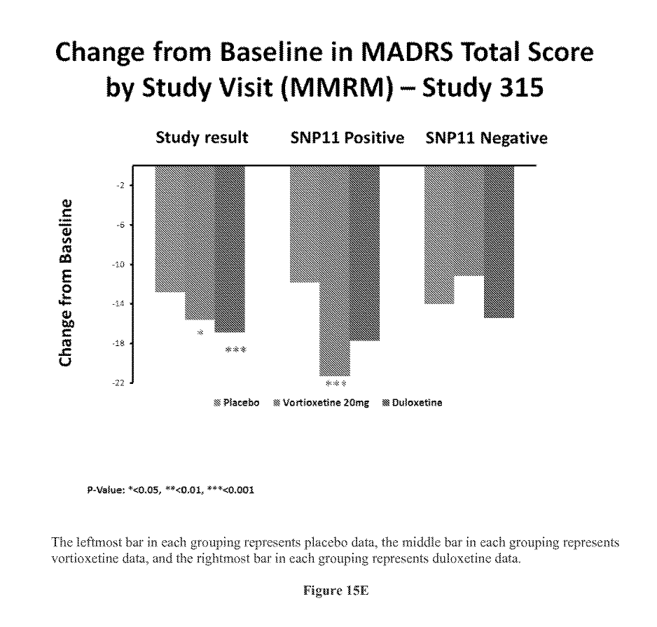

The 10-variant model shows statistically significant evidence for a treatment-specific effect. A subgroup identified via the genetic signature set forth in Table 13 showed statistically significant enhanced treatment effect. See FIG. 14. Moreover, the subjects outside the subgroup showed a statistically significant non-response effect. See FIG. 14.

The subgroup showing enhanced treatment effect was identified using the elastic net/bootstrapping methods set forth in Example 3. In particular, responders were identified using the following formula, where the optimal cutoff was τ*=−0.9:

Scoreᵢ=(rs4045coefficient)*rs4045+(rs59420002coefficient)*rs59420002+(rs7297582coefficient)*rs7297582+(rs2239042coefficient)*rs2239042+(rs7311147coefficient)*rs7311147+(rs12983596coefficient)*rs12983596+(rs9749513coefficient)*rs9749513+(rs62104612coefficient)*rs62104612+(rs1998609coefficient)*rs1998609+(rs4142192coefficient)*rs4142192

The specific algorithm used in this example was as follows:

Scoreᵢ=−1.0*rs4045+1.5*rs59420002−0.3*rs7297582−0.7*rs2239042+0.5*rs7311147−0.5*rs12983596−0.4*rs9749513+0.6*rs62104612−0.9*rs1998609+0.5*rs4142192

A change in MADRS total score was determined via MMRM in patients identified as responders and non-responders using the 10-variant model. FIGS. 14A-F show a change from baseline in MADRS total score in 3 separate arms of the study following administration of duloxetine, 10 mg vortioxetine, and/or 20 mg vortioxetine. In the figures, “SNP10 positive” corresponds to patients in the subgroup and “SNP10 negative” corresponds to patients not in the subgroup.

### Example 10

Table 14 shows genes and gene combinations whose expression levels can be combined in multigene models that significantly correlate with overall response rate in adult MDD patients treated with 20 mg/day vortioxitene. In the table, the B allele represents the minor allele, and the A allele represents the major allele.

TABLE 14
11 variants used in genetic signature

| | | | 95% CI | B | A | G = 0 | G = 1 | G = 2 |
|---|---|---|---|---|---|---|---|---|
| # | dbSNP ID | Gene | Coefficient β range | Allele | Allele | (AA) | (AB) | (BB) |
| 1 | rs4045 | EMID2 | −2.144 to −0.339 | A | G | GG | AG | AA |
| 2 | rs59420002 | CSMD1 | 0.438 to 8.403 | G | A | AA | GA | GG |
| 3 | rs7297582 | CACNA1C | −0.873 to 0.574 | T | C | CC | TC | TT |
| 4 | rs2239042 | CACNA1C | −1.637 to −0.008 | G | A | AA | GA | GG |
| 5 | rs7311147 | CACNA1C | −0.041 to 1.208 | G | A | AA | GA | GG |
| 6 | rs12983596 | ZSCAN4 | −1.615 to 0.657 | C | T | TT | CT | CC |
| 7 | rs9749513 | ZSCAN4 | −1.773 to 0.415 | C | T | TT | CT | CC |
| 8 | rs62104612 | DYM | 0.133 to 1.513 | A | G | GG | AG | AA |
| 9 | rs1998609 | Intergenic | −2.267 to −0.531 | C | T | TT | CT | CC |
| 10 | rs145136593 | LINC00348 | 0.000 to 10.679 | A | G | GG | AG | AA |
| 11 | rs116191388 | FOXL2NB | 0.000 to 9.841 | A | G | GG | AG | AA |

The 11-variant model shows statistically significant evidence for a treatment-specific effect. A subgroup identified via the genetic signature set forth in Table 14 showed statistically significant enhanced treatment effect. Moreover, the subjects outside the subgroup showed a statistically significant non-response effect.

The subgroup showing enhanced treatment effect was identified using the elastic net/bootstrapping methods set forth in Example 3. In particular, responders were identified using the following formula, where the optimal cutoff was τ*=−1.3:

Scoreᵢ=(rs4045coefficient)*rs4045+(rs59420002coefficient)*rs59420002+(rs7297582coefficient)*rs7297582+(rs2239042coefficient)*rs2239042+(rs7311147coefficient)*rs7311147+(rs12983596coefficient)*rs12983596+(rs9749513coefficient)*rs9749513+(rs62104612coefficient)*rs62104612+(rs1998609coefficient)*rs1998609+(rs145136593coefficient)*rs145136593+(rs116191388coefficient)*rs116191388

The specific algorithm used in this example was as follows:

Scoreᵢ=−1.1*rs4045+2.2*rs59420002−0.1*rs7297582−0.8*rs2239042+0.5*rs7311147−0.4*rs12983596−0.5*rs9749513+0.7*rs62104612−1.2*rs1998609+2.5*rs145136593+1.4*rs116191388

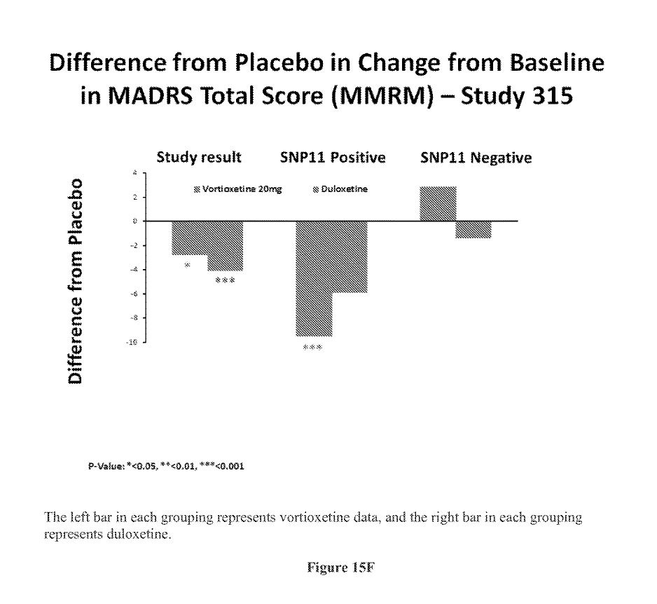

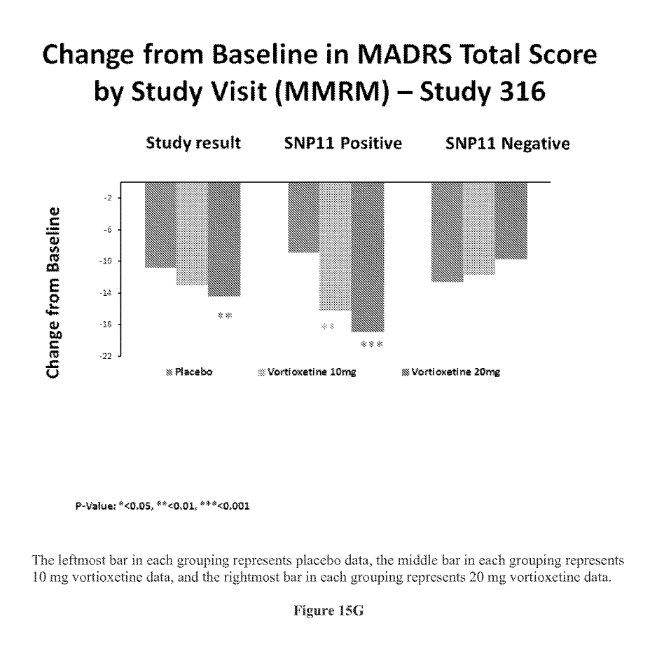

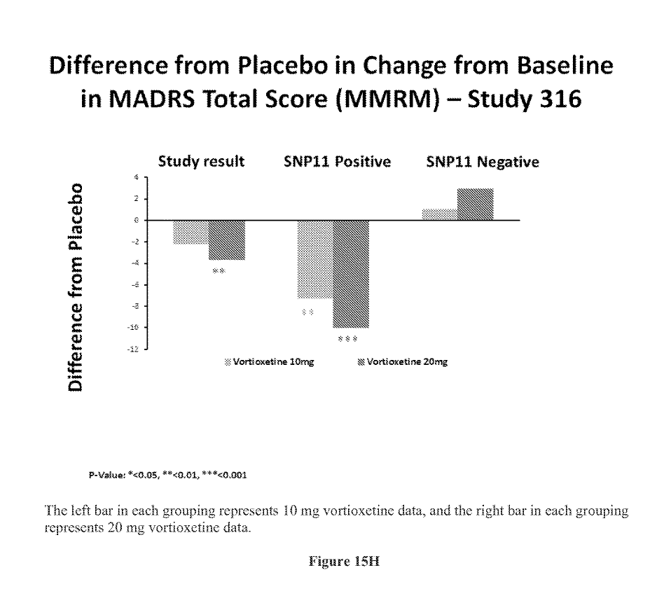

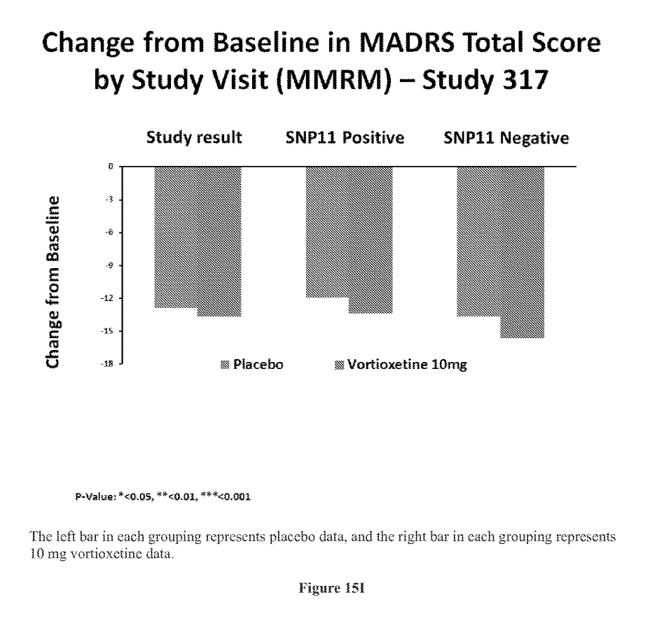

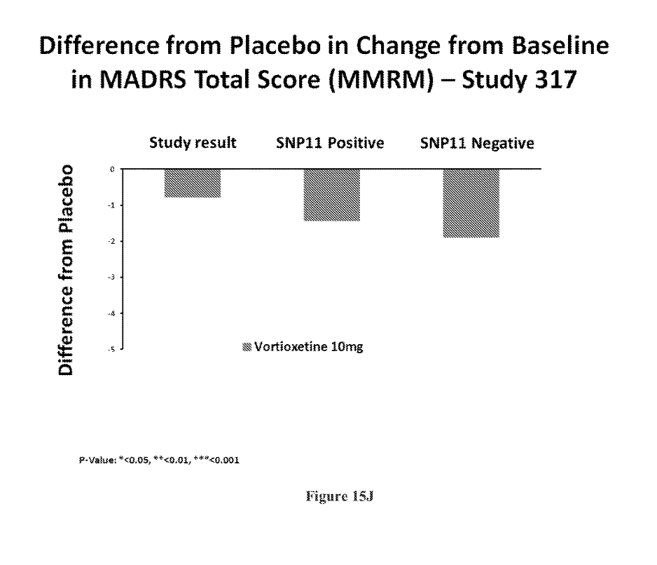

A change in baseline MADRS scores at week 8 and at each visit were determined via MMRM (FIGS. 15A-D). A comparison of the change from baseline in MADRS total score is shown in FIGS. 15E-J. In the figures “SNP11 Positive” corresponds to patients in the subgroup and “SNP11 Negative” corresponds to patients not in the subgroup.

1. A method for treating (i) depression and/or MDD and/or (ii) cognitive impairment in an individual, comprising administering vortioxetine to an individual identified as (i) COL26A1 rs4045 positive, (ii) CACNA1C variant positive, (iii) CSMD1 variant positive, (iv) ZSCAN4 variant positive, (v) ZNF551 variant positive, (vi) COL26A1 rs4045 positive and CACNA1C variant positive (vii) COL26A1 rs4045, CACNA1C, and CSMD1 variant positive, (viii) COL26A1 rs4045, CACNA1C, CSMD1, and ZSCAN4 variant positive, (ix) COL26A1 rs4045, CACNA1C, CSMD1, ZSCAN4, and ZNF551 variant positive, (x) COL26A1 rs4045, CACNA1C, CSMD1, ZSCAN4, DYM, and intergenic variant positive, or (xi) COL26A1 rs4045, CACNA1C, CSMD1, ZSCAN4, DYM, LINC00348, FOXL2NB, and intergenic variant positive.

2. The method of claim 1, wherein the individual suffers from a major depressive disorder (MDD).

3. The method of claim 1, comprising determining that the individual is homozygous for COL26A1 rs4045.

4. The method of claim 1, wherein the individual is heterozygous or homozygous for the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant.

5. (canceled)

6. The method of claim 1, wherein the individual is COL26A1 rs4045, CACNA1C, and CSMD1 variant positive.

7. (canceled)

8. (canceled)

9. The method of claim 1, wherein the individual has rs4045, rs59420002, rs7297582, rs2239042, and rs7311147 variants.

10. The method of claim 1, wherein the individual has rs4045, rs59420002, rs7297582, rs2239042, rs7311147, rs12983596, and rs9749513 variants.

11. The method of claim 1, wherein the individual has rs4045, rs59420002, rs7297582, rs2239042, rs7311147, rs9304796, rs73064580, rs12983596, rs12984275, rs9749513, rs12609579, rs4239480, rs9676604, and rs12162232 variants.

12. The method of claim 9, wherein the individual has one or more of rs9304796, rs73064580, rs12983596, rs12984275, rs9749513, rs12609579, rs4239480, rs9676604, rs12162232, rs10417057, rs10403851, rs56066537, rs112783430, rs9749360, and rs12162230.

13. (canceled)

14. (canceled)

15. (canceled)

16. (canceled)

17. A method for determining the likelihood that an individual suffering from (i) depression and/or MDD and/or (ii) cognitive impairment will experience an enhanced treatment effect when treated with vortioxetine and/or respond favorably to treatment with vortioxetine comprising: assaying a biological sample from the individual for the presence or absence of COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual; and determining if the individual is likely to experience an enhanced treatment effect when treated with vortioxetine when the COL26A1 rs4045 and/or the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant are detected in the sample.

18. The method of claim 17, wherein the individual has a clinical diagnosis of a major depressive disorder (MDD).

19. The method of claim 1 or 17, wherein the CACNA1C variant is selected from the group consisting of rs7297992, rs7297582, rs2239042, rs3819532, rs2239079, rs2239080, kgp5074525, rs4765961, kgp1052923, kgp1390211, rs7311147, rs12312322, rs2108636, rs2238043, rs7295089, kgp3964892, rs10848664, kgp2586442, rs4765700, rs2238095, rs12312322, rs7972947, rs10848664, rs2370602, and combinations thereof.

20. The method of claim 1 or 17, wherein the CACNA1C variant is selected from the group consisting of rs7297582, rs2239042, rs7311147, and combinations thereof.

21. The method of claim 17, wherein the sample is selected from the group consisting of a body fluid sample, a tissue sample, cells and isolated nucleic acids.

22. The method of claim 21, wherein the isolated nucleic acids comprise DNA or RNA.

23. (canceled)

24. The method of claim 17, wherein the assaying comprises reverse transcribing the RNA to produce cDNA.

25. The method of claim 17, comprising detecting the presence of COL26A1 rs4045 and/or a CACNA1C variant and/or a CSMD1 variant and/or a ZSCAN4 variant and/or a ZNF551 variant and/or a DYM variant and/or a LINC00348 variant and/or a FOXL2NB variant and/or an intergenic variant in nucleic acids from the individual.

26. The method of claim 17, comprising determining that the individual is homozygous for COL26A1 rs4045.

27. The method of claim 26, comprising determining that the individual is heterozygous or homozygous for the CACNA1C variant and/or the CSMD1 variant and/or the ZSCAN4 variant and/or the ZNF551 variant and/or the DYM variant and/or the LINC00348 variant and/or the FOXL2NB variant and/or the intergenic variant.

28. (canceled)

29. The method of claim 1 or 17, wherein the CSMD1 variant is rs59420002.

30. The method of claim 1 or 17, wherein the ZSCAN4 variant is selected from the group consisting of rs9304796, rs73064580, rs12983596, rs12984275, rs9749513, rs12609579, rs4239480, rs9676604, rs12162232, rs10417057, rs10403851, rs56066537, rs112783430, rs9749360, and combinations thereof.

31. The method of claim 30, wherein the ZSCAN4 variant is rs12983596 and/or rs9749513.

32. The method of claim 1 or 17, wherein the ZNF551 variant is rs12162230.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. A kit comprising: (i) at least one pair of primers that specifically hybridizes to a genetic variant independently selected from the group consisting of rs4045, rs59420002, rs7297582, rs2239042, and rs7311147, and (ii) a detectably labeled probe that hybridizes to the genetic variant.

82. The kit of claim 81, wherein the kit comprises: a pair of primers that specifically hybridizes to rs4045; a pair of primers that specifically hybridizes to rs59420002; a pair of primers that specifically hybridizes to rs7297582; a pair of primers that specifically hybridizes to rs2239042; and a pair of primers that specifically hybridizes to rs7311147.

83. The kit of claim 81, wherein the kit further comprises (i) at least one pair of primers that specifically hybridizes to a genetic variant independently selected from the group consisting of rs7297992, rs7297582, rs2239042, rs3819532, rs2239079, rs2239080, kgp5074525, rs4765961, kgp1052923, kgp1390211, rs7311147, rs12312322, rs2108636, rs2238043, rs7295089, kgp3964892, rs10848664, kgp2586442, rs4765700, rs2238095, rs12312322, rs7972947, rs10848664, and rs2370602 (ii) a pair of primers that specifically hybridizes to rs59420002, (iii) at least one pair of primers that specifically hybridizes to a genetic variant independently selected from the group consisting of rs9304796, rs73064580, rs12983596, rs12984275, rs9749513, rs12609579, rs4239480, rs9676604, rs12162232, rs10417057, rs10403851, rs56066537, rs112783430, and rs9749360, (iv) a pair of primers that specifically hybridizes to rs59420002, (v) at least one pair of primers that specifically hybridizes to a genetic variant independently selected from the group consisting of rs9304796, rs73064580, rs12983596, rs12984275, rs9749513, rs12609579, rs4239480, rs9676604, rs12162232, rs10417057, rs10403851, rs56066537, rs112783430, and rs9749360, (vi) a pair of primers that specifically hybridizes to rs12162230, (vii) a pair of primers that specifically hybridizes to rs62104612, (viii) a pair of primers that specifically hybridizes to rs145136593, (ix) a pair of primers that specifically hybridizes to rs116191388, and/or (x) at least one pair of primers that specifically hybridizes to a genetic variant independently selected from the group consisting of rs1998609 and rs4142192.

84. (canceled)

85. (canceled)

86. (canceled)

87. (canceled)

88. (canceled)

89. (canceled)

90. (canceled)

91. The kit of claim 81, wherein the kit comprises: a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; and a pair of primers that specifically hybridizes to rs9749513.

92. The kit of claim 81, wherein the kit comprises: a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; a pair of primers that specifically hybridizes to rs9749513; a pair of primers that specifically hybridizes to rs62104612; a pair of primers that specifically hybridizes to rs1998609; and a pair of primers that specifically hybridizes to rs4142192.

93. The kit of claim 81, wherein the kit comprises: a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs12983596; a pair of primers that specifically hybridizes to rs9749513; a pair of primers that specifically hybridizes to rs62104612; a pair of primers that specifically hybridizes to rs1998609; a pair of primers that specifically hybridizes to rs145136593; and a pair of primers that specifically hybridizes to rs116191388.

94. The kit of claim 81, wherein the kit comprises: a pair of primers that specifically hybridizes to rs4045; a pair of primers that specifically hybridizes to rs59420002; a pair of primers that specifically hybridizes to rs7297582; a pair of primers that specifically hybridizes to rs2239042; a pair of primers that specifically hybridizes to rs7311147; a pair of primers that specifically hybridizes to rs9304796; a pair of primers that specifically hybridizes to 73064580; a pair of primers that specifically hybridizes to rs12983596; a pair of primers that specifically hybridizes to rs12984275; a pair of primers that specifically hybridizes to rs9749513; a pair of primers that specifically hybridizes to rs12609579; a pair of primers that specifically hybridizes to rs4239480; a pair of primers that specifically hybridizes to rs9676604; and a pair of primers that specifically hybridizes to rs12162232.


SEQUENCE LISTING (1 sequence)

SEQ ID NO: 1 - DNA, 51 bp, Homo sapiens
tgttctgttttggcctccgaactccraggagtgagtgagaagaactccctg